SEQUENCE LISTING (7 sequences)

SEQ ID NO: 1 - DNA, 407 bp, Pseudomonas aeruginosa
gcacaacaacaataacaagcaacgacgaagacaataaaaacaacacgtaacgactccagcacaacaaaaacaaaatcgcggaggcgcagctaactgattcttttggagaggagttgctgtcgggacccgtcccgcagccagtcggaagaagaataaaactgccttgaggcagcgcacagactggttggatcgctcgacgatcatggcagcatcagcgaccaaagcaatccgtttgctattgaactcccagcctgggagatatccctgaagcgactggctcaagggacgggtcgacaaacaaaaacaacaagcccgaaatcataataaaaacaaagcacgcacctacttgggggggagcttcggctcccccagtagcttcaccccctccctccgttttccccgttttt

SEQ ID NO: 2 - DNA, 16 bp, Pseudomonas aeruginosa
acaacaacaataacaa

SEQ ID NO: 3 - DNA, 11 bp, Pseudomonas aeruginosa
aacaagaacaa

SEQ ID NO: 4 - DNA, 12 bp, Pseudomonas aeruginosa
agaacaacaaaa

SEQ ID NO: 5 - DNA, 13 bp, Pseudomonas aeruginosa
acaacaagaacaa

SEQ ID NO: 6 - DNA, 13 bp, Pseudomonas aeruginosa
agaacaagaacaa

SEQ ID NO: 7 - protein, 11 aa, Artificial Sequence
  TAT cell-penetrating peptide
YGRKKRRQRRR

(19) United States
(12) Patent Application Publication
(10) Pub. No.: US 2023/0149561 A1
(43) Pub. Date: May 18, 2023

(54) Functionalized Nanoparticles and Their Use in Treating Bacterial Infections

(71) Applicant: The Board of Trustees of the Leland Stanford Junior University, Stanford, CA (US)

(72) Inventors: Laurent Bekale, Redwood City, CA (US); Peter Luke Santa Maria, Redwood City, CA (US)

(21) Appl. No.: 17/624,487

(22) PCT Filed: Jul. 10, 2020
(86) PCT No.: PCT/US2020/041667

§ 371 Date: Jan. 3, 2022

Related U.S. Application Data
(60) Provisional application No. 62/873,717, filed on Jul. 12, 2019.

(51) Int. Cl. A61K 47/69; A61K 47/62; A61P 31/04; B82Y 5/00

(52) U.S. Cl. CPC A61K 47/6929; A61K 47/62; A61P 31/04; B82Y 5/00

(57) ABSTRACT

Compositions, methods, and kits are provided for treating bacterial infections with functionalized nanoparticles. Recalcitrant infections are often difficult to treat because of the presence of persister cells, a subpopulation of bacterial cells that is highly tolerant of traditional antibiotics. Persister cells are dormant, which makes them less susceptible to many antibiotics, which are designed to kill growing cells. Administration of nanoparticles by themselves or in combination with one or more antibiotics was found to be highly efficacious in eradicating persister cells and for treating infections for a broad range of bacterial species, including Gram-positive and Gram-negative bacteria. Such treatment was effective not only in eradicating plank-tonic bacteria but also bacteria in biofilms.

### BACKGROUND

Antibiotics are the mainstay of modern clinical medicine. However, bacteria develop resistance to both natural and synthetic antibiotics within years of their first clinical use (Walsh (2003) Nature Reviews Microbiology 1:65-70). Current mechanisms of antibiotic resistance include: decreased uptake by changes in outer membrane permeability; antibiotic excretion by activation of efflux pump-proteins; enzymatic modification of the antibiotic; modification of antibiotic targets; and bacterial physiology such as biofilm (van Hoek et al. (2011) Front Microbiol 2:203).

In the United States and Europe alone, over 50,000 people die every year because of resistant infections (The Review on Antimicrobial Resistance. Antimicrobial Resistance: Tackling a crisis for the health and wealth of nations (2014), amr-review.org/Publications.html)). Lengths of stays in a hospital are prolonged by antibiotic-resistant infections, and these same infections are often acquired in hospitals. The economic impact of antibiotic resistant infections is estimated to be between US $5 billion and US $24 billion per year in the United States alone (Hall (2004) Nature Reviews Microbiology 2:430-435). However, the drug pipelines of pharmaceutical companies have not kept pace with the evolution of antibiotic resistance. In 2004, only 1.5% of all the drugs in development by the world's 15 largest pharmaceutical companies were antibiotics (Smith and Coast, “The economic burden of antimicrobial resistance: why it is more serious than current studies suggest.” (2012), researchgate.net/publication/291413454). The new reality that we must face is that the pharmaceutical companies are not presently aligned for the discovery of new antibiotics. A strategy to protect our existing antibiotics is through the use of antibiotic adjuvants, compounds that enhance the activity of current drugs and minimize, and even directly block resistance (Lu et al. (2009) Proc. Natl. Acad. Sci. U.S.A. 106(12):4629-4634, Gonzalez-Bello (2017) Bioorg. Med. Chem. Lett. 27(18):4221-4228). Another strategy is the used of ant-virulence agents. These agents can circumvent antibiotic resistance by disarming pathogens of virulence factors that facilitate human disease while leaving bacterial growth pathways (Dickey et al. (2017) Nat. Rev. Drug Discov. 16(7):457-471).

Bacterial cells, attached to a surface, can aggregate to each other to form biofilms. Bacteria growing biofilms may exhibit increased tolerance to antimicrobial agents, it is very difficult or eliminate substantially reduce. Biofilm bacteria have two dormant phenotypes: the viable but non-culturable (VBNC) state and the persister state. Dormant phenotypes (VBNC and persisters) allow bacteria to survive in conditions that are deadly to the rest of their genetically identical lineage. Once in biofilms, they can escape the immune system. Thus, one of the main roles of biofilm is to provide a protective habitat for persisters and VBNC by shielding them from the immune system (Lewis (2010) Microbe (Washington, D.C.) 5(10):429-437). Another property of biofilms is their capacity to be more resistant to antimicrobial agents than planktonic cells (Spoering et al. (2001) J. Bacteriol. 183(23):6746-6751). Thus, there is an ongoing and unmet need for an improved approach to treating antibiotic resistant infections.

### SUMMARY

Compositions, methods, and kits are provided for treating bacterial infections with nanoparticles. Recalcitrant infections are often difficult to treat because of the presence of persister cells, a subpopulation of bacterial cells that is highly tolerant of traditional antibiotics. Persister cells are dormant, which makes them less susceptible to many antibiotics, which are designed to kill growing cells. Administration of nanoparticles in combination with one or more antibiotics for treating an infection is highly efficacious in eradicating persister cells and is effective against both planktonic bacteria as well as bacteria in biofilms for a broad range of bacterial species, including Gram-positive and Gram-negative bacteria. In particular, the formulations comprising nanoparticles described herein are useful for enhancing the effect of antibiotics as well as reducing the virulence of bacteria.

In one aspect, a nanoparticle is provided having a size of less than 10 nm in length that is functionalized with an anionic moiety and a cell penetrating peptide, wherein the anionic moiety and the cell penetrating peptide are attached to the outer surface of the nanoparticle.

Exemplary cell penetrating peptides include, without limitation, HIV-Tat, penetratin, transportan, octaarginine, nonaarginine, antennapedia, TP10, Buforin II, MAP (model amphipathic peptide), K-FGF, Ku70, mellittin, pVEC, Pep-1, SynB1, Pep-7, CADY, GALA, pHLIP, KALA, R7W, and HN-1, which can readily transport nanoparticles across plasma membranes.

The anionic moiety may include for example, without limitation, a carboxylate functional group, a phosphate functional group, or a sulfate functional group.

In certain embodiments, the nanoparticle further comprises a polyethylene glycol (PEG) polymer, wherein the PEG polymer is attached to the outer surface of the nanoparticle. In some embodiments, the PEG polymer is functionalized with the anionic moiety. For example, the PEG polymer may be functionalized with an acid moiety. In some embodiments, the PEG polymer comprises a carboxylate group (e.g., PEG carboxylic acid (PEG-COOH), hydroxyl PEG carboxylic acid, PEG-acetic acid, PEG glutaric acid, PEG succinic acid, PEG glutaramide acid, PEG succinamide acid). In some embodiments, the PEG polymer is functionalized with a thiol group and an anionic moiety (e.g., thiol-carboxyl polyethylene glycol (COOH-PEG-SH)). In other embodiments, the PEG polymer is functionalized with a cationic moiety such as an amine group (PEG-NH₂) or a neutral moiety such as a hydroxyl group (PEG-OH).

In certain embodiments, the nanoparticle is further functionalized with a D-carbohydrate including, without limitation, D-glucose, D-mannitol, D-arabinose, or D-xylose.

In certain embodiments, the nanoparticle is further functionalized with a D-amino acid including, without limitation, D-glutamic acid, D-leucine, D-methionine, D-tyrosine and D-tryptophan.

In certain embodiments, the nanoparticle is further functionalized with a nucleic acid comprising a CrcZ RNA sequence or a CrcZ A-rich motif sequence. In certain embodiments, the CrcZ RNA sequence comprises the nucleotide sequence of SEQ ID NO:1, or a or sequence displaying at least about 80-100% sequence identity thereto, including any percent identity within this range, such as 81, 82, 83, 84, 85, 86, 87, 88, 89, 90, 91, 92, 93, 94, 95, 96, 97, 98, 99% sequence identity thereto; or an RNA equivalent thereof. In certain embodiments, the CrcZ A-rich motif sequence comprises: a) ACAACAACAATAACAA (SEQ ID NO:2); b) CAATAAGAA; c) AACAAGAACAA (SEQ ID NO:3); d) AGAACAACAAAA (SEQ ID NO:4); e) ACAACAAGAACAA (SEQ ID NO:5); f) AGAACAAGAACAA (SEQ ID NO:6); g) AACAACAA; h) AAAAACAA; or i) an RNA equivalent of a)-i).

In certain embodiments, the nanoparticle further comprises an antimicrobial agent having bactericidal activity against persister cells or bacteria residing in biofilms, wherein the antimicrobial agent is attached to the outer surface of the nanoparticle.

In certain embodiments, the nanoparticle further comprises a linker connecting a functionalization agent (e.g., cell penetrating peptide, nucleic acid comprising CrcZ RNA, an antimicrobial agent) to the outer surface of the nanoparticle.

In certain embodiments, the nanoparticle ranges in size from about 1 nm to about 500 nm in length, including any length within this range such as 1, 2, 3, 4, 5, 6, 7, 8, 9, 10, 12, 14, 16, 18, 20, 30, 40, 50, 60, 70, 80, 90, 100, 120, 140, 160, 180, 200, 220, 240, 260, 280, 300, 320, 340, 360, 380, 400, 420, 440, 460, 480, or 500 nm in length. In some embodiments, the nanoparticle is less than 10 nm in length. In some embodiments, the nanoparticles are about 1 to about 2 nm in length.

In certain embodiments, the nanoparticle comprises a metal, a ceramic, graphite, graphene, or other carbon-based material, silica, or boron. For example, the nanoparticle may comprise a metal including, without limitation, one or more of gold, silver, platinum, titanium, palladium, rhodium, ruthenium, tin, nickel, copper, aluminum, or an oxide, carbide, nitride, or an alloy thereof. In certain embodiments, the nanoparticle is biocompatible with human cells.

In another aspect, a composition comprising a nanoparticle described herein is provided. In certain embodiments, the composition further comprises a pharmaceutically acceptable excipient or carrier.

In certain embodiments, the composition further comprises an antibiotic. Exemplary antibiotics include, without limitation, fluoroquinolones, aminoglycosides, penicillins, tetacyclines, cephalosporins, macrolides, sulfonamides, carbapenems, ansamycins, carbacephems, carbapenems, lincosamides, monobactams, and oxazolidinones. For example, the antibiotic may include a fluoroquinolone such as ofloxacin, moxifloxacin, ciprofloxacin, gemifloxacin, levofloxacin, or finafloxacin, or a derivative thereof.

In another aspect, a method of treating an infection in a subject is provided, the method comprising administering a therapeutically effective amount of a composition comprising a functionalized nanoparticle to the subject. In some embodiments, the method further comprises administering a therapeutically effective amount of at least one antibiotic in combination with the composition comprising the nanoparticle.

Exemplary antibiotics include, without limitation, fluoroquinolones, aminoglycosides, penicillins, tetacyclines, cephalosporins, macrolides, sulfonamides, carbapenems, ansamycins, carbacephems, carbapenems, lincosam ides, monobactams, and oxazolidinones. For example, the antibiotic may include a fluoroquinolone such as ofloxacin or a derivative thereof.

In another embodiment, a method of treating an infection in a subject is provided, the method comprising administering a therapeutically effective amount of a nanoparticle in combination with a therapeutically amount of an antibiotic to the subject.

In certain embodiments, the subject has a chronic infection. In some embodiments, the subject has an infection including, without limitation, an ear infection, a cutaneous infection, a lung infection, chronic suppurative otitis media (CSMO), an infection associated with cystic fibrosis, tuberculosis, or an infection in a wound. In some embodiments, the infection is associated with formation of a bacterial biofilm in the subject. In certain embodiments, the infection comprises pathogenic bacteria that are resistant to one or more antibiotics. In some embodiments, the subject has previously been treated for the infection with one or more antibiotics that have not successfully cleared the infection. In another embodiment, the infection is an infection (e.g. Pseudomonas) in a subject who has cystic fibrosis.

In certain embodiments, the treatment eradicates all or most biofilm bacteria and planktonic bacteria. In some embodiments, the treatment eradicates all or most persister cells, which may be, for example, in a biofilm or internalized by a macrophage. In some embodiments, the persister cells that are eradicated by the treatment described herein are multidrug tolerant persister cells. Treatment may eradiate persister cells comprising either Gram-negative or Gram-positive bacteria, including, without limitation, Pseudomonas aeruginosa persister cells.

In certain embodiments, multiple cycles of treatment are administered to the subject. For example, nanoparticles described herein may be administered alone or in combination with an antibiotic either intermittently or according to a daily dosing regimen.

Compositions comprising nanoparticles may be administered by any suitable mode of administration. For example, the composition may be administered intravenously, subcutaneously, by inhalation, or topically. Alternatively, the composition may be administered locally at the site of infected tissue. For example, for an ear infection, the composition comprising nanoparticles may be administered locally into the ear canal.

In another embodiment, a method of eradicating bacteria in a biofilm is provided, the method comprising contacting the biofilm with an effective amount of a composition comprising a functionalized nanoparticle. In some embodiments, the method further comprises contacting the biofilm with an effective amount of at least one antibiotic. The methods described herein may be used to eradicate bacteria, for example, in a biofilm on a medical device, a personal hygiene article, a toiletry, a cosmetic, a disinfectant, a cleaning solution, or in a water treatment or distribution system.

In another embodiment, a method of eradicating dormant bacteria is provided, the method comprising contacting the dormant bacteria with an effective amount of a composition comprising a functionalized nanoparticle. In some embodiments, the method further comprises contacting the dormant bacteria with an effective amount of at least one antibiotic. The dormant bacteria may be present, for example, in a biofilm, in a liquid culture, or on an inanimate surface.

In another embodiment, a method of inhibiting SOS signaling or RecA activity in bacteria, the method comprising contacting bacteria with an effective amount of a composition comprising a functionalized nanoparticle.

In another aspect, a kit is provided comprising a nanoparticle described herein and instructions for treating a bacterial infection. In some embodiments, the kit further comprises an antibiotic including without limitation, a fluoroquinolone such as ofloxacin or a derivative thereof.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIGS. 1A-1D: Ofloxacin fails to eradicate PA CSOM biofilms. FIG. 1A) Comparison of biofilm formed (A₅₉₅/OD₆₀₀) from P. aeruginosa wild-type PA01 with otopathogenic strain (PA CSOM). Amount of crystal violet stained biofilm attached to 96-well microtiter plate (A₅₉₅) normalized by total cell growth (OD₆₀₀) from PA01 and PA CSOM. After a 48-h incubation, the production of biofilm extracellular matrix was measured by crystal violet staining (A₅₉₅). Optical density at 600 nm (OD₆₀₀) from each well was measured to determine the total cell growth. Data are presented as mean±s.d. (n=5). FIGS. 1B-1C) Eradication of PA01 and PA CSOM biofilms by ofloxacin. Following the treatment, the optical density (OD) from recovery plates after 48 h incubation was measured at 650 nm (OD₆₅₀) using a spectrophotometer. Data are presented as mean±s.d. (n=3). FIG. 1D) Representative petri dish showing persister cells resuscitation following ofloxacin treatment. LB agar plate was spotted with recovery media and incubated for 48h. Values refer to the concentration of ofloxacin (μg/mL). MBEC of PAO1=750 ug/ml compared to PA CSOM not susceptible to maximum concentration of ofloxacin (3000 ug/ml). Statistical comparisons were done using a two-tailed t-test as indicated. ***p≤0.001.

FIGS. 2A-2B: Increasing the fraction of persister cells promotes biofilm tolerance to ofloxacin. FIG. 2A) Susceptibility of surviving persister cells in logarithmic-phase culture. Ofloxacin demonstrated sensitivity after persister cells resuscitation in logarithmic-phase. Data are presented as mean±s.d. (n=3). FIG. 2B) Comparison of persister cell fraction between PAO1 and PA CSOM in logarithmic-phase culture. Cells of logarithmic-phase were treated with ofloxacin (100 pg/mL) for 24 h and then plated for colony counting. The persister fraction was calculated as the ratio between the number of surviving bacteria after ofloxacin treatment compared to that measured in the corresponding untreated control (PBS). Data are presented as mean±s.d. (n=3). Statistical comparisons were done using a two-tailed t-test as indicated. ****p≤0.0001 and **p≤0.01.

FIGS. 3A-3B: AuNC@CPP overcome the drug-refractory state associated with biofilm formation. Representative petri dish showing persister cells resuscitation following treatment of 48 h-old PA01 (FIG. 3A) and PA CSOM (FIG. 3B) biofilms by ofloxacin, AuNC@CPP and their combination for 24 h. The biofilm cells are left to recover (48 h) in fresh media without drug and then 5 μL was plated on Luria-Bertani (LB) broth that contains agar. For each condition, the three spots on the petri dishes are the recovery media from three independent experiment (n=3). No growth of PAO1 or PA CSOM is seen with AuNC@CPP combined with ofloxacin (5 or 10 ug/ml).

FIGS. 4A-4D: Oral administration of AuNC@CPP does not cause systemic toxicity. FIG. 4A) Schematic of experimental design. FIGS. 4B-4D) Representative histological photomicrograph of organs by H&E staining. There was no obvious morphologic change on the histological structure of tissues after daily oral gavage at a dose of 10 mg/kg daily for 14 days with AuNC@CPP and PBS. The tissue samples were collected at 35 days post-treatment.

FIGS. 5A-5B: Floxin®Otic plus AuNC@CPP has an antimicrobial activity superior to Floxin®Otic alone in mouse model of chronic P. aeruginosa ear infection mimicking CSOM. FIG. 5A) Schematic of experimental design. FIG. 5B) Comparison of the number of bacteria per milliliter (CFU/mL) from middle ear effusion 14 days after the end of the following treatments: placebo control (phosphate-buffered saline, PBS), FLOXIN®Otic (24 μg of ofloxacin) and combination (24 μg of ofloxacin+296 μg of AuNC@CPP). The CFU/mL from each mouse are plotted as individual points and error bars represent the deviation in CFU/mL within an experimental group. ^{ϕ}indicates that no middle ear effusion was able to be sampled due to technical constraints. There was no difference between the PBS control group and the ofloxacin group. AuNC@CPP plus FLOXIN®Otic combination led to a 5 log reduction in bacteria 14 days after treatment. Statistical comparisons were done using a two-tailed t-test as indicated. *p≤0.05 and Not significant (N.S).

FIG. 6: Mechanism of AuNC@CPP action. Cartoon depicting the molecular mechanisms underlying hypersensitization of persister cells within P. aeruginosa biofilm in the presence of AuNC@CPP.

FIG. 7: Cartoon depicting the flowchart to evaluate the eradication capacities of ofloxacin, AuNC@CPP and their combination towards preformed biofilm using the Calgary biofilm device (The scheme was adapted from Emery Pharma, emerypharma.com/biology/biofilm-eradication/). (1-2) Bacteria culture is prepared and dispensed into a 96-well microplate. (3) The peg lid is placed in the bacteria culture and incubated to generate the biofilm. (4) The peg lid is gently rinsed to removed planktonic bacteria and a serial diluted test solution is dispensed into a new 96-well microplate. (5) The pegs covered in biofilm are incubated in the test solutions. (6) The peg lid is again gently rinsed to remove planktonic bacteria and placed in a new 96-well microplate containing recovery media. The peg lid is then sonicated to dislodge the biofilm into the recovery media. (7) Following sonication, the peg lid is replaced with a regular 96-well microplate lid and the plate containing recovery media is incubated. Following incubation, the OD₆₅₀ absorbance is read on a spectrophotometer. Wells with an OD₆₅₀ of less than 0.1 is evidence of biofilm eradication. (8) Spot plated on LB agar plates is use to confirm biofilm eradication.

FIG. 8: Cartoon depicting the flowchart to evaluate ofloxacin sensitivity to persister cells resuscitation in logarithmic-phase culture. (1) Surviving persister cells following the treatment of PA CSOM biofilm with ofloxacin at 3000 μg/mL were replaced in fresh media and incubated at 37° C. and were aerated at 225 r.p.m. (2) Cells in logarithmic-phase (OD₆₀₀=0.3) were treated with either phosphate-buffered saline (PBS) or ofloxacin (100 μg/mL) for 24 h. (3) following the treatment, cells were plated to determine the number of bacteria per milliliter (CFU/mL).

FIG. 9: Eradication of PA01 biofilm by AuNC@CPP alone. Following the treatment, the optical density (OD) from recovery plates after 48h incubation was measured at 650 nm (OD₆₅₀) using a spectrophotometer. Data are presented as mean±s.d. (n=3).

FIG. 10: Cartoon depicting the synthesis of engineered gold nanocluster (AuNC@CPP). a) UV-vis absorption spectra of the as-prepared AuNC@CPP. b) Diameter by DLS measurement. c) Surface charge by DLS measurement.

FIG. 11: Cytotoxic effects of AuNC@CPP. A549 cells were treated with AuNC@CPP for 24 h and the cell viability was determined by MTT assay. Cells exposed with AuNC@CPP at 3200 μg/mL shows more than 90% of viable cells. Data are presented as mean±s.d. (n=4).

FIGS. 12A-12H: Evolution of the body weight in healthy mice after oral gavage at a dose of 10 mg/kg for 14 days with AuNC@CPP and PBS. C57BL/6J mice were treated with 10 mg/kg every day for 14 days and changes in the body weight of healthy mice at 35 days was evaluated in male (FIG. 12A) and female (FIG. 12B) mice, and organs, including thymus (FIG. 12C), kidney (FIG. 12D), heart (FIG. 12E), spleen (FIG. 12F), liver (FIG. 12G), and testis (FIG. 12H). *indicates statistically significant difference versus PBS (p<0.05) of treated samples versus control (PBS). Data are presented as mean±s.d.

FIGS. 13A-13B: AuNC@CPP restores antibiotic susceptibility of persister cells. (FIG. 13A) Comparison of the biofilm eradication capacities of ofloxacin, AuNC@CPP (800 μg/mL) and combination. Following treatment, optical density (OD) measured from recovery plates after 48h incubation (650 nm). Combination eradicated persister cells of Pseudomonas aeruginosa from a CF patient. All data (n=3). Wells with OD₆₅₀ of <0.1 is evidence of biofilm eradication. (FIG. 13B) Human alveolar cells exhibited no change in viability upon exposure to AuNC@CPP concentrations.

FIG. 14: Intracellular ofloxacin (OFL) contents of stationary phase planktonic PA in the absence and presence of AuNC@CPP. The Y axis shows the OFL uptake of the expressed as a percentage (%) increase in fluorescence over untreated stationary phase planktonic PA. The data represent the average values of three experiments.

FIGS. 15A-15D: Evidence of persister cell eradication obtained through in vitro testing. Persister cells were treated with ofloxacin alone (FIG. 15A), AuNC@CPP alone (FIG. 15B), or a combination of OFL and AuNC@CPP (FIG. 15C). FIG. 15D shows that AuNC addition enhances ofloxacin effectiveness and results in eradication of both PA biofilms and late stationary phase planktonic cells. Minimum biofilm eradication concentration (MBEC). The averages of data from three experiments with six replicates per experiment are shown. Insert pictures show the LB-agar plates of the survivor persister cells that have formed colonies after 48 hr incubation.

FIG. 16: Viability of E. coli biofilm determined by the Calgary Biofilm Device (CBD) after treatment of AuNC@PEG-NH₂ and AuNC@PEG-OH for 24 h. Following treatment, optical density (OD) from recovery plates after 48 h incubation was measured at 650 nm. Wells with an OD650 of ≤0.1 is evidence of biofilm eradication. The photographs show the spot plated on LB agar plates and the values on the image represent the concentration of AuNC@PEG-NH₂ and AuNC@PEG-OH in μg/mL. The minimum E. coli biofilm eradication concentration (MBEC) value of both AuNCs is 1 mg/mL. All data represent the mean±SD of 3 replicates.

FIG. 17: Demonstration of AuNC@CPP effectiveness in SA CSOM biofilm eradication. Commercial fluoroquinolones (Ofloxacin, OFL) are available at 3000 ug/ml. This is unable to eradicate biofilms of a SA clinical isolate from CSOM SA. Combined with AUNC@CPP, OFL is effective at eradicating SA biofilms.

FIG. 18: Representative petri dish showing persister cells resuscitation following treatment of 48h-old PA01 and PA CSOM biofilms by AuNC@CPP for 24 h. The biofilm cells are left to recover (48 h) in fresh media without AuNC@CPP and then 5 μL was plated on Luria-Bertani (LB) broth that contains agar. The three spots on the petri dishes are the recovery media from three independent experiment (n=3). The MBEC of AuNC@CPP against PA01 and PA CSOM biofilm is 1600 and 3200 ug/ml, respectively.

FIG. 19: Viability of the E. coli biofilm determined by the Calgary Biofilm Device (CBD) after treatment of AuNC@PEG-NH₂ and AuNC@PEG-OH for 24 h. Following treatment, optical density (OD) from recovery plates after 48 h incubation was measured at 650 nm. Wells with an OD650 of 0.1 is evidence of biofilm eradication. The photographs show the spot plated on LB agar plates and the values on the image represent the concentration of AuNC@PEG-NH₂ and AuNC@PEG-OH in pg/mL. The minimum E. coli biofilm eradication concentration (MBEC) value of both AuNCs is 1 mg/mL. All data represent the mean±SD of 3 replicates.

FIG. 20: Cell-penetrating peptide alone cannot eradicate the biofilm; the entire entity of AuNCs was required for biofilm eradication. The photograph shows the spot plated on LB agar plates and the values on the image represent the concentration of cell-penetrating peptide (CPP) in pg/mL. The CPP did not show any biofilm eradication at the concentration up to 4000 μg/mL. Each spot represents an independent experiment (n=3).

### DETAILED DESCRIPTION OF EMBODIMENTS

Compositions comprising functionalized nanoparticles and methods of using them in treating bacterial infections are provided. In particular, functionalized nanoparticles are useful for treating chronic infections associated with production of bacterial biofilms, which are not responsive to conventional antibiotic treatment. Without being bound by theory, bacteria in biofilms tend to be more resistant to treatment with antibiotics, in part, because the biofilm extracellular matrix and outer layers of cells protect bacterial cells in the interior. In addition, many bacterial cells in a biofilm adopt a dormant phenotype, becoming metabolically inactive, which makes them less susceptible to antibiotics that need to be metabolized in order to be effective (e.g., penicillin requires cell wall remodeling in an active bacterial cell in order to cause cell death). Dormant cells in biofilms, which have entered a non-growing or extremely slow-growing physiological state, and as a result have become resistant to antimicrobial drugs, are referred to herein as “persister cells” because of their ability to persist after other active bacterial cells have been eradicated by the immune system or antimicrobial agents. Persister cells are often associated with chronic infections because of the difficulty of eradicating them with conventional antibiotic treatment. The methods described herein are especially useful for treating chronic infections to render persister cells in biofilms more susceptible to antibiotic treatment.

Before the present compositions comprising functionalized nanoparticles and methods of using them in treating bacterial infections are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a bacterial cell” includes a plurality of such bacterial cells and reference to “the nanoparticle” includes reference to one or more nanoparticles and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “nanoparticle” refers to an organic, inorganic, or hybrid nanoparticle having a size ranging from about 1 nm to about 500 nm in length. Nanoparticles may have dimensions of 500 nm or less, including 250 nm or less, or 200 nm or less, or 150 nm or less, or 100 nm or less, or 50 nm or less, or 40 nm or less, or 30 nm or less, or 25 nm or less, or 20 nm or less, or 15 nm or less, or 10 nm or less, or 5 nm or less, or 4 nm or less, or 3 nm or less, or 2 nm or less, or 1 nm or less. In some instances, the nanoparticle has dimensions of 2 nm or less.

The term “persister cells” refers to cells that have entered a non-growing (i.e., dormant) or extremely slow-growing physiological state that renders them less susceptible or resistant to antimicrobial drugs. Such cells may “persist” after planktonic bacterial cells have been eradicated by the immune system or conventional treatment with an antimicrobial agent. Persister cells are commonly found in biofilms.

As used herein, the term “antimicrobial agent” is interchangeable with the term “antibiotic” and refers to any agent capable of having bactericidal or bacterial static effects on growth. Antibiotics include, but are not limited to, a β-lactam antibiotic, an aminoglycoside, an aminocyclitol, a quinolone, a tetracycline, a macrolide, a lincosamide, a glycopeptide, a lipopeptide, a polypeptide antibiotic, a sulfonamide, trimethoprim, chloramphenicol, isoniazid, a nitroimidazole, a rifampicin, a nitrofuran, methenamine, and mupirocin.

The term “anti-bacterial effect” means the killing of, or inhibition or stoppage of the growth and/or reproduction of bacteria.

The term “efflux pump” as used herein refers to a protein assembly, which transports or exports substrate molecules from the cytoplasm or periplasm of a cell, in an energy-dependent or independent fashion. The term “efflux pump activity” as used herein refers to a mechanism responsible for export of substrate molecules, including antimicrobial agents, outside the cell. The term “efflux pump inhibitor” as used herein refers to a compound, which interferes with the ability of an efflux pump to transport or export a substrate, including antimicrobial agent.

The term “CrcZ” as used herein encompasses all forms of CrcZ and also includes biologically active fragments, for example, including one or more CrcZ A-rich motifs, variants, analogs, and derivatives thereof that retain biological activity (e.g., disrupting or interfering with bacterial biofilm formation).

A CrcZ RNA, DNA, nucleic acid, polynucleotide, or oligonucleotide refers to a molecule derived from any species of CrcZ-expressing bacteria. The molecule need not be physically derived from bacteria, but may be synthetically or recombinantly produced. A number of CrcZ nucleic acid sequences are known. Representative sequences of CrcZ (SEQ ID NO:1) and CrcZ A-rich motifs (SEQ ID NOS:2-6) from Pseudomonas aeruginosa are presented in the Sequence Listing. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to construct functionalized nanoparticles for treating a bacterial infection, as described herein.

By “fragment” is intended a molecule consisting of only a part of the intact full-length sequence and structure. For a nucleic acid, the fragment can include a 5′ deletion a 3′ deletion, and/or an internal deletion of the nucleic acid. Active fragments of a particular nucleic acid will generally include at least about 5-16 contiguous nucleotides of the full length molecule, but may include at least about 8-20 contiguous nucleotides of the full length molecule, and can include at least about 20-50 or more contiguous nucleotides of the full length molecule, or any integer between 5 nucleotides and the full length sequence, provided that the fragment in question retains biological activity (e.g., the ability to eradicate a bacterial infection). For a protein or peptide, the fragment can include a C-terminal deletion an N-terminal deletion, and/or an internal deletion of the polypeptide. Active fragments of a particular protein or peptide will generally include at least about 5-14 contiguous amino acid residues of the full length molecule, but may include at least about 15-25 contiguous amino acid residues of the full length molecule, and can include at least about 20-50 or more contiguous amino acid residues of the full length molecule, or any integer between 5 amino acids and the full length sequence, provided that the fragment in question retains biological activity (e.g., the ability to eradicate a bacterial infection).

The term “treatment” as used herein refers to either (1) the prevention of infection or reinfection (prophylaxis), or (2) the reduction or elimination of symptoms of an infectious disease of interest (therapy).

By “therapeutically effective dose or amount” of nanoparticles is intended an amount that, when administered alone or in combination with an antibiotic, as described herein, brings about a positive therapeutic response, such as improved recovery from an infection, including any infection caused by Gram-positive or Gram-negative bacteria. Additionally, a therapeutically effective dose or amount may eradicate persister cells as well as other bacterial cells, including planktonic bacteria as well as bacteria in biofilms. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

“Substantially purified” generally refers to isolation of a component such as a substance (compound, nanoparticle, nucleic acid, polynucleotide, RNA, DNA, protein, or polypeptide) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography, gel filtration, and sedimentation according to density.

By “isolated” is meant, when referring to a polypeptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The terms “recipient”, “individual”, “subject”, “host”, and “patient”, are used interchangeably herein and refer to any vertebrate subject for whom diagnosis, treatment, or therapy is desired, particularly humans. By “vertebrate subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

“Biocompatible” generally refers to a material and any metabolites or degradation products thereof that are generally non-toxic to the recipient and do not cause any significant adverse effects to the subject.

“Homology” refers to the percent identity between two polynucleotide or two polypeptide molecules. Two nucleic acid, or two polypeptide sequences are “substantially homologous” to each other when the sequences exhibit at least about 50% sequence identity, preferably at least about 75% sequence identity, more preferably at least about 80% 85% sequence identity, more preferably at least about 90% sequence identity, and most preferably at least about 95% 98% sequence identity over a defined length of the molecules. As used herein, substantially homologous also refers to sequences showing complete identity to the specified sequence.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353 358, National biomedical Research Foundation, Washington, D.C., which adapts the local homology algorithm of Smith and Waterman Advances in Appl. Math. 2:482 489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages, the Smith Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “derived from” is used herein to identify the original source of a molecule but is not meant to limit the method by which the molecule is made which can be, for example, by chemical synthesis or recombinant means.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

The term “hydrophilic polymer” refers to a material that has the property of dissolving in, absorbing, or mixing easily with water, and comprises repeating units constituting a molecular weight of at least 200 up to 8,000 or more. Hydrophilic polymers include, without limitation, polyethylene glycol (PEG) as well as other materials, which can be used to solubilize nanoparticles. Materials for this purpose include polyethylene glycol (PEG), polyoxyethylene, polymethylene glycol, polytrimethylene glycols, polyvinyl-pyrrolidones, poly lysine (D or L) and derivatives, and polyoxyethylene-polyoxypropylene block polymers and copolymers. The hydrophilic polymers can be linear or multiply branched, and may include multi-arm block copolymers. The hydrophilic polymer renders the nanoparticles soluble when attached thereto in sufficient numbers.

### Methods

Compositions comprising functionalized nanoparticles and methods of using them in treating bacterial infections are provided. In particular, functionalized nanoparticles are useful for treating chronic infections associated with production of bacterial biofilms, which are not responsive to conventional antibiotic treatment. Without being bound by theory, bacteria in biofilms tend to be more resistant to treatment with antibiotics, in part, because the biofilm extracellular matrix and outer layers of cells protect bacterial cells in the interior. In addition, many bacterial cells in a biofilm adopt a dormant phenotype, becoming metabolically inactive, which makes them less susceptible to antibiotics that need to be metabolized in order to be effective (e.g., penicillin requires cell wall remodeling in an active bacterial cell in order to cause cell death). Dormant cells in biofilms, which have entered a non-growing or extremely slow-growing physiological state, and as a result have become resistant to antimicrobial drugs, are referred to herein as “persister cells” because of their ability to persist after other active bacterial cells have been eradicated by the immune system and antimicrobial agents. Persister cells are often associated with chronic infections because of the difficulty of eradicating them with conventional antibiotic treatment. The methods described herein are especially useful for treating chronic infections to render persister cells in biofilms more susceptible to antibiotic treatment.

### Functionalized Nanoparticles for Treatment of Bacterial Infections

Nanoparticles may be functionalized with one or more agents, including polymers (e.g., PEGylated nanoparticles), cell transduction peptides (e.g., TAT), anti-microbial agents, and/or bacterial RNAs (e.g., CrcZ) that enhance delivery and/or the effectiveness of the nanoparticles in eradicating bacteria. The functionalized nanoparticle may be an organic, inorganic, or hybrid nanoparticle having a size ranging from about 1 nm to about 500 nm in length. Nanoparticles may have dimensions of 500 nm or less, including 250 nm or less, or 200 nm or less, or 150 nm or less, or 100 nm or less, or 50 nm or less, or 40 nm or less, or 30 nm or less, or 25 nm or less, or 20 nm or less, or 15 nm or less, or 10 nm or less, or 5 nm or less, or 4 nm or less, or 3 nm or less, or 2 nm or less, or 1 nm or less. In some instances, the nanoparticle has dimensions of 2 nm or less.

The nanoparticle is typically spherical in shape, but nanoparticles having other shapes may also be used. For example, the nanoparticle may have a shape such as, but not limited to, an ellipsoid, a rod, a cone, a cube, a cuboid (e.g., a rectangular box), a pyramid, or an irregular shape, etc. In certain instances, combinations of different shapes of nanoparticles may be included in a composition. In some embodiments, the nanoparticle is substantially spherical in shape, and thus may have dimensions measured as a diameter of a sphere. For example, nanoparticles may have an average diameter of 500 nm or less, including 250 nm or less, or 200 nm or less, or 150 nm or less, or 100 nm or less, or 50 nm or less, or 40 nm or less, or 30 nm or less, or 25 nm or less, or 20 nm or less, or 15 nm or less, or 10 nm or less, or 5 nm or less, or 4 nm or less, or 3 nm or less, or 2 nm or less, or 1 nm or less. In some instances, a substantially spherical nanoparticle has an average diameter of 2 nm or less.

The nanoparticle may comprise, for example, a metal, a ceramic, carbon-based nanomaterials, silicon or silica, boron, polymers, lipids, or proteins. In certain embodiments, the nanoparticle is composed of an oxide of silicon, aluminum, a transition metal (e.g., titanium, zirconium, and the like), aluminosilicate, boron nitride, or a combination thereof. Exemplary materials that may be used in nanoparticles include, but are not limited to, silicon dioxide (e.g., silica), titanium dioxide, silicon-aluminum-oxide, aluminum oxide, and iron oxide. In some embodiments, the nanoparticle comprises a metal including, without limitation, one or more of gold, silver, platinum, titanium, palladium, rhodium, ruthenium, tin, nickel, copper, aluminum, or an oxide, carbide, nitride, or alloy thereof. In some instances, the nanoparticle is composed of other inorganic materials, such as, but not limited to, diatomaceous earth, calcium hydroxyapatite, and the like. Nanoparticles may also be composed of hydrophobic polymers such as, but not limited to, polylactide; polylactic acid; polyolefins, such as polyethylene, poly(isobutene), poly(isoprene), poly(4-methyl-1-pentene), polypropylene, ethylene-propylene copolymers, and ethylenepropylene-hexadiene copolymers; ethylene-vinyl acetate copolymers; and styrene polymers, such as poly(styrene), poly(2-methylstyrene), styrene-acrylonitrile copolymers, and styrene-2,2,3,3,-tetrafluoro-propyl methacrylate copolymers. Nanoparticles may also be composed of natural polymers such as proteins, including, without limitation, albumin, silk, keratin, collagen, elastin, corn zein, and soy protein-based nanoparticles; or polysaccharide-based polymers, including, without limitation, chitosan, hyaluronic acid, alginate, glucan, dextran, and cyclodextrin-based nanoparticles. Carbon-based nanoparticles may include, without limitation, carbon nanotubes, graphite, graphene, fullerenes and nanodiamonds. Combinations of the above materials may also be included in nanoparticles. In certain embodiments, the nanoparticle is biocompatible with human cells.

In some embodiments, the outer surface of a nanoparticle is functionalized with an anionic moiety. The anionic moiety may include, for example, without limitation, a carboxylate functional group, a phosphate functional group, or a sulfate functional group.

In some embodiments, the outer surface of a nanoparticle is functionalized with a hydrophilic polymer to solubilize the nanoparticle. Exemplary polymers that can be used for this purpose include, without limitation, polyethylene glycol (PEG), polyoxyethylene, polymethylene glycol, polytrimethylene glycols, polyvinyl-pyrrolidones, polylysine (D or L) and derivatives, and polyoxyethylene-polyoxypropylene block polymers and copolymers. The hydrophilic polymers can be linear or multiply branched, and may include multi-arm block copolymers. The hydrophilic polymer renders the nanoparticles soluble when attached thereto in sufficient numbers. Additionally, a polymer may also protect nanoparticles from protein adsorption and reduce immunological reactions to the nanoparticles, which helps to prolong their stability in the bloodstream.

In some embodiments, the outer surface of the nanoparticle is functionalized with a polyethylene glycol (PEG) polymer (i.e., PEGylated nanoparticle). The PEG polymer may be branched or unbranched. In some cases, the PEG polymer has an average molecular mass of 1000 Da or more, such as 1500 Da or more, including 2000 Da or more, or 3000 Da or more, or 4000 Da or more, or 5000 Da or more, or 6000 Da or more, or 7000 Da or more, or 8000 Da or more, or 9000 Da or more, or 10,000 Da or more, or 15,000 Da or more, or 20,000 Da or more. In certain instances, the PEG polymer has an average molecular mass of 2000 Da. In certain instances, the PEG polymer has an average molecular mass of 2000 Da.

In some embodiments, the PEG polymer is functionalized with the anionic moiety. For example, the PEG polymer may be functionalized with an acid moiety. In some embodiments, the PEG polymer comprises a carboxylate group (e.g., PEG carboxylic acid (PEG-COOH), hydroxyl PEG carboxylic acid, PEG-acetic acid, PEG glutaric acid, PEG succinic acid, PEG glutaramide acid, PEG succinamide acid). In other embodiments, the PEG polymer is functionalized with a cationic moiety such as an amine group (PEG-NH₂) or a neutral moiety such as a hydroxyl group (PEG-OH).

In certain embodiments, the nanoparticle further comprises a D-carbohydrate including, without limitation, D-glucose, D-mannitol, D-arabinose, or D-xylose, wherein the D-carbohydrate is attached to the outer surface of the nanoparticle.

In certain embodiments, the nanoparticle further comprises a D-amino acid including, without limitation, D-glutamic acid, D-leucine, D-methionine, D-tyrosine and D-tryptophan, wherein the D-amino acid is attached to the outer surface of the nanoparticle.

In certain embodiments, the nanoparticle is linked to an internalization sequence, a protein transduction domain, or a cell penetrating peptide to facilitate entry into a cell. Cell penetrating peptides that can be used include, but are not limited to, HIV-Tat, penetratin, transportan, octaarginine, nonaarginine, antennapedia, TP10, Buforin II, MAP (model amphipathic peptide), K-FGF, Ku70, mellittin, pVEC, Pep-1, SynB1, Pep-7, CADY, GALA, pHLIP, KALA, R7W, and HN-1, which can readily transport nanoparticles across plasma membranes (see, e.g., Jones et al. (2012) “Cell entry of cell penetrating peptides and tales of tails wagging dogs,” J. Control Release. 2012, in press; Fonseca et al. (2009) Adv. Drug Deliv. Rev. 61(11):953-64; Schwarze et al. (1999) Science. 285(5433):1569-72; Derossi et al. (1996) J. Biol. Chem. 271(30):18188-18193; Fuchs et al. (2004) Biochemistry 43(9):2438-2444; and Yuan et al. (2002) Cancer Res. 62(15):4186-4190).

In certain embodiments, the nanoparticle is functionalized with a nucleic acid comprising a CrcZ RNA sequence or a biologically active fragment thereof, for example, including one or more CrcZ A-rich motifs, or a variant, analog, or derivative thereof that retains biological activity (e.g., disrupting or interfering with bacterial biofilm formation). CrcZ RNA sequences may be derived from any bacterial species expressing CrcZ. A number of CrcZ nucleic acid sequences are known. Representative sequences of CrcZ (SEQ ID NO:1) and CrcZ A-rich motifs (SEQ ID NOS:2-6) from Pseudomonas aeruginosa are presented in the Sequence Listing. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, can be used to construct functionalized nanoparticles for treating a bacterial infection, as described herein.

### Conjugation

Surface functionalization of nanoparticles may be performed by any method known in the art. Functionalization of a nanoparticle involves conjugation of an agent (e.g., PEG, CrcZ, cell penetrating peptides, anionic moiety, and/or other agents or moieties) to a molecule on the outer surface of the nanoparticle. A surface coating may be applied to nanoparticles to introduce functional groups to facilitate attachment of agents. For example, gold nanoparticles with surface coatings comprising thiol, carboxyl, amine, aldehyde, hydroxyl, or azide groups, PEG, dextran, streptavidin, or maleimide and compounds to facilitate bioconjugation are commercially available from a number of companies (e.g., SigmaAldrich (St. Louis, Mo.), and Cytodiagnostics (Burlington, Ontario, Canada), Creative Diagnostics (Shirley, N.Y.), and Nanocs (New York, N.Y.)). An agent may be conjugated to a nanoparticle directly or indirectly through a linker. Exemplary linkers include, without limitation, thioC6 linker (thiohexyl), PEG polymers, diethylenetriaminepentaacetic acid (DTPA), 1,4,7,10-tetraazacyclododecane-1,4,7,10-tetraacetic acid (DOTA), and hydrazide compounds. For a discussion of bioconjugation techniques, see, e.g., Chemistry of Bioconjugates: Synthesis, Characterization, and Biomedical Applications (R. Narain ed., Wiley, 2014), G. T. Hermanson Bioconjugate Techniques (Academic Press, 3^{rd} edition, 2013), and Bioconjugation Protocols: Strategies and Methods (Methods in Molecular Biology, S. S. Mark ed., Humana Press, 2^{nd} edition, 2011), Avvakumova et al. (2014) Trends Biotechnol. 32(1):11-20., Couto et al. (2017) Crit Rev Biotechnol. 37(2):238-250, Sivaram et al. (2018) Adv. Healthc Mater. 7(1), van Vught et al. (2014) Comput Struct Biotechnol J. 9:e201402001; Massa et al. (2016) Expert Opin Drug Deliv 13:1-15; Yeh et al. (2015) PLoS One 10(7):e0129681; Freise et al. (2015) Mol Immunol. 67(2 Pt A):142-152; herein incorporated by reference in their entireties.

A variety of conjugation methods and chemistries can be used to conjugate agents to a nanoparticle. Various zero-length, homo-bifunctional, and hetero-bifunctional crosslinking reagents can be used. Zero-length crosslinking reagents include direct conjugation of two intrinsic chemical groups with no introduction of extrinsic material. Agents that catalyze formation of a disulfide bond belong to this category. Another example is reagents that induce condensation of a carboxyl and a primary amino group to form an amide bond such as carbodiimides, ethylchloroformate, Woodward's reagent K (2-ethyl-5-phenylisoxazolium-3′-sulfonate), and carbonyldiimidazole. Homo- and hetero-bifunctional reagents generally contain two identical or two non-identical sites, respectively, which may be reactive with amino, sulfhydryl, guanidino, indole, or nonspecific groups.

Suitable amino-reactive groups include, but are not limited to, N-hydroxysuccinimide (NHS) esters, imidoesters, isocyanates, acylhalides, arylazides, p-nitrophenyl esters, aldehydes, and sulfonyl chlorides. Suitable sulfhydryl-reactive groups include, but are not limited to, maleimides, alkyl halides, pyridyl disulfides, and thiophthalimides. In other embodiments, carbodiimides soluble in both water and organic solvent, are used as carboxyl-reactive reagents. These compounds react with free carboxyl groups forming a pseudourea that can then couple to available amines, yielding an amide linkage.

in some embodiments, an agent is conjugated to a nanoparticle using a homobifunctional crosslinker. In some embodiments, the homobifunctional crosslinker is reactive with primary amines. Homobifunctional crosslinkers that are reactive with primary amines include NHS esters, imidoesters, isothiocyanates, isocyanates, acylhalides, arylazides, p-nitrophenyl esters, aldehydes, and sulfonyl chlorides. Non-limiting examples of homobifunctional NHS esters include disuccinimidyl glutarate (DSG), disuccinimidyl suberate (DSS), bis(sulfosuccinimidyl)suberate (BS), disuccinimidyl tartarate (DST), disulfosuccinimidyl tartarate (sulfo-DST), bis-2-(succinimidooxycarbonyloxy)ethylsulfone (BSOCOES), bis-2-(sulfosuccinimidooxycarbonyloxy)ethylsulfone (sulfo-BSOCOES), ethylene glycolbis(succinimidylsuccinate) (EGS), ethylene glycolbis(sulfosuccinimidylsuccinate) (sulfo-EGS), dithiobis(succinimidylpropionate (DSP), and dithiobis(sulfosuccinimidylpropionate (sulfo-DSP). Non-limiting examples of homobifunctional imidoesters include dimethyl malonimidate (DMM), dimethyl succinimidate (DMSC), dimethyl adipimidate (DMA), dimethyl pimelimidate (DMP), dimethyl suberimidate (DMS), dimethyl-3,3′-oxydipropionimidate (DODP), dimethyl-3,3′-(methylenedioxy)dipropionimidate (DM DP), dimethyl-,3′-(dimethylenedioxy)dipropionimidate (DDDP), dimethyl-3,3′-(tetramethylenedioxy)dipropionimidate (DTDP), and dimethyl-3,3′-dithiobispropionimidate (DTBP).

Non-limiting examples of homobifunctional isothiocyanates include: p-phenylenediisothiocyanate (DITC), and 4,4′-diisothiocyano-2,2′-disulfonic acid stilbene (DI DS). Non-limiting examples of homobifunctional isocyanates include xylene-diisocyanate, toluene-2,4-diisocyanate, toluene-2-isocyanate-4-isothiocyanate, 3-methoxydiphenylmethane-4,4′-diisocyanate, 2,2′-dicarboxy-4,4′-azophenyldiisocyanate, and hexamethylenediisocyanate. Non-limiting examples of homobifunctional arylhalides include 1,5-difluoro-2,4-dinitrobenzene (DFDNB), and 4,4′-difluoro-3,3′-dinitrophenyl-sulfone. Non-limiting examples of homobifunctional aliphatic aldehyde reagents include glyoxal, malondialdehyde, and glutaraldehyde. Non-limiting examples of homobifunctional acylating reagents include nitrophenyl esters of dicarboxylic acids. Non-limiting examples of homobifunctional aromatic sulfonyl chlorides include phenol-2,4-disulfonyl chloride, and alpha-naphthol-2,4-disulfonyl chloride. Non-limiting examples of additional amino-reactive homobifunctional reagents include erythritolbiscarbonate, which reacts with amines to give biscarbamates.

In some embodiments, the homobifunctional crosslinker is reactive with free sulfhydryl groups. Homobifunctional crosslinkers reactive with free sulfhydryl groups include, e.g., maleimides, pyridyl disulfides, and alkyl halides. Non-limiting examples of homobifunctional maleimides include bismaleimidohexane (BMH), N,N′-(1,3-phenylene)bismaleimide, N,N′-(1,2-phenylene)bismaleimide, azophenyldimaleimide, and bis(N-maleimidomethyl)ether. Non-limiting examples of homobifunctional pyridyl disulfides include 1,4-di-3′-(2′-pyridyldithio)propionamidobutane (DPDPB). Non-limiting examples of homobifunctional alkyl halides include 2,2′-dicarboxy-4,4′-diiodoacetamidoazobenzene, α,α′-diiodo-p-xylenesulfonic acid, α, α′-dibromo-p-xylenesulfonic acid, N,N′-bis(b-bromoethyl)benzylamine, N,N′-di(bromoacetyfiphenylhydrazine, and 1,2-di(bromoacetyfiamino-3-phenylpropane.

In some embodiments, an agent is conjugated to a nanoparticle using a heterobifunctional reagent. Suitable heterobifunctional reagents include amino-reactive reagents comprising a pyridyl disulfide moiety; amino-reactive reagents comprising a maleimide moiety; amino-reactive reagents comprising an alkyl halide moiety; and amino-reactive reagents comprising an alkyl dihalide moiety.

Non-limiting examples of hetero-bifunctional reagents with a pyridyl disulfide moiety and an amino-reactive NHS ester include N-succinimidyl-3-(2-pyridyldithio)propionate (SPDP), succinimidyl 6-3-(2-pyridyldithio)propionamidohexanoate (LC-SPDP), sulfosuccinimidyl 6-3-(2-pyridyldithio)propionamidohexanoate (sulfo-LCSPDP), 4-succinimidyloxycarbonyl-α-methyl-α-(2-pyridyldithio)toluene (SMPT), and sulfosuccinimidyl 6-α-methyl-α-(2-pyridyldithio)toluamidohexanoate (sulfo-LC-SMPT).

Non-limiting examples of heterobifunctional reagents comprising a maleimide moiety and an amino-reactive NHS ester include succinimidyl maleimidylacetate (AMAS), succinimidyl 3-maleimidylpropionate (BMPS), N-gamma-maleimidobutyryloxysuccinimide ester (GMBS)N-gamma-maleimidobutyryloxysulfosuccinimide ester (sulfo-GMBS) succinimidyl 6-maleimidylhexanoate (EMCS), succinimidyl 3-maleimidylbenzoate (SMB), m-maleimidobenzoyl-N-hydroxysuccinimide ester (MBS), m-maleimidobenzoyl-N-hydroxysulfosuccinimide ester (sulfo-MBS), succinimidyl 4-(N-maleimidomethylicyclohexane-1-carboxylate (SMCC), sulfosuccinimidyl 4-(N-maleimidomethyl)cyclohexane-1-carboxylate (sulfo-SMCC), succinimidyl 4-(p-maleimidophenyl)butyrate (SMPB), and sulfosuccinimidyl 4-(p-maleimidophenyl)butyrate (sulfo-SMPB).

Non-limiting examples of heterobifunctional reagents comprising an alkyl halide moiety and an amino-reactive NHS ester include N-succinimidyl-(4-iodoacetyl)aminobenzoate (SIAB), sulfosuccinimidyl-(4-iodoacetyl)aminobenzoate (sulfo-SIAB), succinimidyl-6-(iodoacetyl)aminohexanoate (SIAX), succinimidyl-6-(6-((iodoacetyl)-amino)hexanoylamino)hexanoate (SIAXX), succinimidyl-6-(((4-(iodoacetyl)-amino)methyl)-cyclohexane-1-carbonyl)aminohexanoate (SIACX), and succinimidyl-4((iodoacetyl)-amino)methylcyclohexane-1-carboxylate (SIAC).

A non-limiting example of a hetero-bifunctional reagent comprising an amino-reactive NHS ester and an alkyl dihalide moiety is N-hydroxysuccinimidyl 2,3-dibromopropionate (SDBP). A non-limiting example of a hetero-bifunctional reagent comprising an alkyl halide moiety and an amino-reactive p-nitrophenyl ester moiety includes p-nitrophenyl iodoacetate (NPIA).

In another example, a 3-ThioC6 linker can be used to functionalize an agent with a thiol group to facilitate attachment to nanoparticles or other agents. For example, the 3-ThioC6 linker can be used to add a thiol group to the 3′ terminus of a nucleic acid comprising a CrcZ RNA sequence or a CrcZ A-rich motif sequence. The free thiol can be used as a reactive functional group to attach maleimide compounds or for conjugation through disulfide linkages.

An alternative bioconjugation method uses click chemistry. Click chemistry reactions include the Huisgen 1,3-dipolar cycloaddition copper catalyzed reaction (Tornoe et al., 2002, J Organic Chem 67:3057-64), cycloaddition reactions such as Diels-Alder reactions, nucleophilic substitution reactions (especially to small strained rings like epoxy and aziridine compounds), reactions involving formation of urea compounds, and reactions involving carbon-carbon double bonds, such as alkynes in thiol-yne reactions. See, e.g., Kolb et al., 2004, Angew Chem Int Ed 40:3004-31; Evans, 2007, Aust J Chem 60:384-95; Millward et al. (2013) Integr Biol (Camb) 5(1):87-95), Lallana et al. (2012) Pharm Res 29(1):1-34, Gregoritza et al. (2015) Eur J Pharm Biopharm. 97(Pt B):438-453, Musumeci et al. (2015) Curr Med Chem. 22(17):2022-2050, McKay et al. (2014) Chem Biol 21(9):1075-1101, Ulrich et al. (2014) Chemistry 20(1):34-41, Pasini (2013) Molecules 18(8):9512-9530, and Wangler et al. (2010) Curr Med Chem. 17(11):1092-1116; herein incorporated by reference in their entireties.

### Pharmaceutical Compositions

Functionalized nanoparticles, as described herein, can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition can also include an antimicrobial agent for preventing or deterring microbial growth. Nonlimiting examples of antimicrobial agents include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the nanoparticles or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the nanoparticles (e.g., when contained in a drug delivery system) in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, ocular, or localized delivery.

The pharmaceutical preparations herein can also be housed in a syringe, an implantation device, or the like, depending upon the intended mode of delivery and use. Preferably, the compositions comprising nanoparticles are in unit dosage form, meaning an amount of a composition appropriate for a single dose, in a premeasured or pre-packaged form.

The compositions herein may optionally include one or more additional agents, such as antibiotics, adjuvants, immunostimulatory agents, vaccines, and/or other medications used to treat a subject for an infection. Compounded preparations may include nanoparticles and one or more other agents for treating an infection, such as, but not limited to, antibiotics including broad spectrum, bactericidal, or bacteriostatic antibiotics such as penicillins including penicillin G, penicillin V, procaine penicillin, benzathine penicillin, veetids (Pen-Vee-K), piperacillin, pipracil, pfizerpen, temocillin, negaban, ticarcillin, and Ticar; penicillin combinations such as amoxicillin/clavulanate, augmentin, ampicillin/sulbactam, unasyn, piperacillin/tazobactam, zosyn, ticarcillin/clavulanate, and timentin; tetacyclines such as chlortetracycline, doxycycline, demeclocycline, eravacycline, lymecycline, meclocycline, methacycline, minocycline, omadacycline, oxytetracycline, rolitetracycline, sarecycline, tetracycline, and tigecycline; cephalosporins such as cefacetrile (cephacetrile), cefadroxil (cefadroxyl; duricef), cefalexin (cephalexin; keflex), cefaloglycin (cephaloglycin), cefalonium (cephalonium), cefaloridine (cephaloradine), cefalotin (cephalothin; keflin), cefapirin (cephapirin; cefadryl), cefatrizine, cefazaflur, cefazedone, cefazolin (cephazolin; ancef, kefzol), cefradine (cephradine; velosef), cefroxadine, ceftezole, cefaclor (ceclor, distaclor, keflor, raniclor), cefonicid (monocid), cefprozil (cefproxil; cefzil), cefuroxime (zefu, zinnat, zinacef, ceftin, biofuroksym, xorimax), cefuzonam, loracarbef (lorabid) cefbuperazone, cefmetazole (zefazone), cefminox, cefotetan (cefotan), cefoxitin (mefoxin), cefotiam (pansporin), cefcapene, cefdaloxime, cefdinir (sefdin, zinir, omnicef, kefnir), cefditoren, cefetamet, cefixime (fixx, zifi, suprax), cefmenoxime, cefodizime, cefotaxime (claforan), cefovecin (convenia), cefpimizole, cefpodoxime (vantin, pecef, simplicef), cefteram, ceftamere (enshort), ceftibuten (cedax), ceftiofur (naxcel, excenel), ceftiolene, ceftizoxime (cefizox), ceftriaxone (rocephin), cefoperazone (cefobid), ceftazidime (meezat, fortum, fortaz), latamoxef (moxalactam), cefclidine, cefepime (maxipime), cefluprenam, cefoselis, cefozopran, cefpirome (cefrom), cefquinome, flomoxef, ceftobiprole, ceftaroline, ceftolozane, cefaloram, cefaparole, cefcanel, cefedrolor, cefempidone, cefetrizole, cefivitril, cefmatilen, cefmepidium, cefoxazole, cefrotil, cefsumide, ceftioxide, cefuracetime, and nitrocefin; quinolones//fluoroquinolones such as flumequine (Flubactin), oxolinic acid (Uroxin), rosoxacin (Eradacil), cinoxacin (Cinobac), nalidixic acid (NegGam, Wintomylon), piromidic acid (Panacid), pipemidic acid (Dolcol), ciprofloxacin (Zoxan, Ciprobay, Cipro, Ciproxin), fleroxacin (Megalone, Roquinol), lomefloxacin (Maxaquin), nadifloxacin (Acuatim, Nadoxin, Nadixa), norfloxacin (Lexinor, Noroxin, Quinabic, Janacin), ofloxacin (Floxin, Oxaldin, Tarivid), pefloxacin (Peflacine), rufloxacin (Uroflox), enoxacin (Enroxil, Penetrex), balofloxacin (Baloxin), grepafloxacin (Raxar), levofloxacin (Cravit, Levaquin), pazufloxacin (Pasil, Pazucross), sparfloxacin (Zagam), temafloxacin (Omniflox), tosufloxacin (Ozex, Tosacin), clinafloxacin, gatifloxacin (Zigat, Tequin, Zymar-ophthalmic), moxifloxacin (Avelox,Vigamox), sitafloxacin (Gracevit), prulifloxacin (Quisnon), besifloxacin (Besivance), delafloxacin (Baxdela), gemifloxacin (Factive) and trovafloxacin (Trovan), ozenoxacin, danofloxacin (Advocin, Advocid), difloxacin (Dicural, Vetequinon), enrofloxacin (Baytril), ibafloxacin (Ibaflin), marbofloxacin (Marbocyl, Zenequin), orbifloxacin (Orbax, Victas), and sarafloxacin (Floxasol, Saraflox, Sarafin); macrolides such as azithromycin, clarithromycin, erythromycin, fidaxomicin, telithromycin, carbomycin A, josamycin, kitasamycin, midecamycin/midecamycin acetate, oleandomycin, solithromycin, spiramycin, troleandomycin, tylosin/tylocine, roxithromycin, telithromycin, cethromycin, solithromycin, tacrolimus, pimecrolimus, sirolimus, amphotericin B, nystatin, and cruentaren; sulfonamides such as sulfonamide, sulfacetamide, sulfadiazine, sulfadimidine, sulfafurazole (sulfisoxazole), sulfisomidine (sulfaisodimidine), sulfamethoxazole, sulfamoxole, sulfanitran, sulfadimethoxine, sulfamethoxypyridazine, sulfametoxydiazine, sulfadoxine, sulfametopyrazine, and terephtyl; aminoglycosides such as kanamycin A, amikacin, tobramycin, dibekacin, gentamicin, sisomicin, netilmicin, neomycins B, C, neomycin E (paromomycin), streptomycin, plazomicin, amikin, garamycin, kantrex, neo-fradin, netromycin, nebcin, humatin, spectinomycin(Bs), and trobicin; carbapenems such as imipenem, meropenem, ertapenem, doripenem, panipenem/betamipron, biapenem, tebipenem, razupenem (PZ-601), lenapenem, tomopenem, and thienamycin (thienpenem); ansamycins such as geldanamycin, herbimycin, rifaximin, and xifaxan; carbacephems such as loracarbef and lorabid; carbapenems such as ertapenem, invanz, doripenem, doribax, imipenem/cilastatin, primaxin, meropenem, and merrem; glycopeptides such as teicoplanin, targocid, vancomycin, vancocin, telavancin, vibativ, dalbavancin, dalvance, oritavancin, and orbactiv; lincosamides such as clindamycin, cleocin, lincomycin, and lincocin; lipopeptides such as daptomycin and cubicin; macrolides such as azithromycin, zithromax, sumamed, xithrone, clarithromycin, biaxin, dirithromycin, dynabac, erythromycin, erythocin, erythroped, roxithromycin, troleandomycin, tao, telithromycin, ketek, spiramycin, and rovamycine; monobactams such as aztreonam and azactam; nitrofurans such as furazolidone, furoxone, nitrofurantoin, macrodantin, and macrobid; oxazolidinones such as linezolid, zyvox, vrsa, posizolid, radezolid, and torezolid; polypeptides such as bacitracin, colistin, coly-mycin-S, and polymyxin B; drugs against mycobacteria such as clofazimine, lamprene, dapsone, avlosulfon, capreomycin, capastat, cycloserine, seromycin, ethambutol, myambutol, ethionamide, trecator, isoniazid, I.N.H., pyrazinamide, aldinamide, rifampicin, rifadin, rimactane, rifabutin, mycobutin, rifapentine, priftin, and streptomycin; and other antibiotics such as arsphenamine, salvarsan, chloramphenicol, chloromycetin, fosfomycin, monurol, monuril, fusidic acid, fucidin, metronidazole, flagyl, mupirocin, bactroban, platensimycin, quinupristin/dalfopristin, synercid, thiamphenicol, tigecycline, tigacyl, tinidazole, tindamax fasigyn, trimethoprim, proloprim, and trimpex; adjuvants, including aluminum salts (alum), such as aluminum hydroxide, aluminum phosphate, aluminum sulfate, etc.; oil-in-water emulsion formulations; (saponin adjuvants; Complete Freund's Adjuvant (CFA) and Incomplete Freund's Adjuvant (IFA); cytokines, such as interleukins (IL-1, IL-2, IL-4, IL-5, IL-6, IL-7, IL-12, interferons, macrophage colony stimulating factor (M-CSF), tumor necrosis factor (TNF), etc.; detoxified mutants of a bacterial ADP-ribosylating toxin such as a cholera toxin (CT), pertussis toxin (PT), or an E. coli heat-labile toxin (LT); oligonucleotides comprising CpG motifs; as well as other immunostimulatory molecules; and vaccines against bacteria and infectious diseases, including any vaccine comprising bacterial antigenic proteins or attenuated or dead bacteria and, optionally, adjuvants for boosting an immune response against bacteria, such as vaccines against tuberculosis, diphtheria, tetanus, pertussis, Haemophilus influenzae type B, cholera, typhoid, Streptococcus pneumoniae, and the like.

Alternatively, such agents can be contained in a separate composition from the composition comprising the nanoparticles and co-administered concurrently, before, or after the composition comprising the nanoparticles.

### Administration

At least one therapeutically effective cycle of treatment with a composition comprising nanoparticles, as described herein, will be administered to a subject for treatment of a bacterial infection. Bacterial infections that can be treated by the methods described herein include bacterial infections caused by Gram negative bacteria such as, but not limited to, Acinetobacter (e.g., Acinetobacter baumannii), Actinobacillus, Bordetella, Brucella, Campylobacter, Cyanobacteria, Enterobacter (e.g., Enterobacter cloacae), Erwinia, Escherichia coli, Franciscella, Helicobacter (Helicobacter pylori), Hemophilus (e.g., Hemophilus influenzae), Klebsiella (e.g., Klebsiella pneumoniae), Legionella (e.g., Legionella pneumophila), Moraxella (e.g., Moraxella catarrhalis), Neisseria (e.g., Neisseria gonorrhoeae, Neisseria meningitidis), Pasteurella, Proteus (e.g., Proteus mirabilis), Pseudomonas (e.g., Pseudomonas aeruginosa), Salmonella (e.g., Salmonella enteritidis, Salmonella typhi), Serratia (e.g., Serratia marcescens), Shigella, Treponema, Vibrio (e.g., Vibrio cholerae), and Yersinia (e.g., Yersinia pestis), as well as Gram positive bacteria such as, but not limited to, Actinobacteria, such as Actinomyces (e.g., Actinomyces Arthrobacter, Bifidobacterium, Corynebacterium (e.g., Corynebacterium diphtheriae), Frankia, Micrococcus, Micromonospora, Mycobacterium (e.g., Mycobacterium tuberculosis, Mycobacterium leprae), Nocardia, Propionibacterium, and Streptomyces; Firmicutes, such as Bacilli, order Bacillales including Bacillus, Listeria (e.g., Listeria monocytogenes), and Staphylococcus (e.g., Staphylococcus aureus, Staphylococcus epidermidis), Bacilli (e.g., Bacilli anthracis, Bacilli cereus), order Lactobacillales, including Enterococcus, Lactobacillus, Lactococcus, Leuconostoc, Pediococcus, and Streptococcus (e.g., Streptococcus pneumoniae, Streptococcus mutans, Streptococcus sanguinis, Streptococcus pyogenes), Clostridia (e.g., Clostridioides difficile, Clostridium perfringens, Clostridium botulinum, Clostridium tetani, Clostridium sordellii), including Acetobacterium, Clostridium, Eubacterium, Heliobacterium, Heliospirillum, Megasphaera, Pectinatus, Selenomonas, Zymophilus, and Sporomusa, Mollicutes, including Mycoplasma (e.g., Mycoplasma pneumoniae), Spiroplasma, Ureaplasma, and Erysipelothrix.

By “therapeutically effective dose or amount” of nanoparticles is intended an amount that, when administered alone or in combination with an antibiotic, as described herein, brings about a positive therapeutic response, such as improved recovery from an infection, including any infection caused by Gram-positive or Gram-negative bacteria. Additionally, a therapeutically effective dose or amount may eradicate persister cells as well as other bacterial cells, including planktonic bacteria and bacteria in biofilms. The exact amount required will vary from subject to subject, depending on the species, age, and general condition of the subject, the severity of the condition being treated, the particular type of nanoparticles and their functionalization, other antimicrobial agents or drugs employed in combination, the mode of administration, and the like. An appropriate “effective” amount in any individual case may be determined by one of ordinary skill in the art using routine experimentation, based upon the information provided herein.

In certain embodiments, multiple therapeutically effective doses of compositions comprising nanoparticles, and/or one or more other therapeutic agents, such as antibiotics, adjuvants, immunostimulatory agents, vaccines, and/or other drugs for treating an infection, or other medications will be administered. The compositions comprising nanoparticles are typically, although not necessarily, administered orally, via injection (subcutaneously, intravenously, or intramuscularly), by infusion, topically, or locally. Additional modes of administration are also contemplated, such as intra-arterial, intravascular, pulmonary, intralesional, intraparenchymatous, rectal, transdermal, transmucosal, intrathecal, intraocular, intraperitoneal, and so forth.

The preparations according to the invention are also suitable for local treatment. For example, compositions comprising nanoparticles may be administered directly to the site of infected tissue. The particular preparation and appropriate method of administration can be chosen to target the nanoparticles to sites of chronic infection and sites of bacterial biofilms where persister cells typically reside and require eradication.

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising nanoparticles and/or other agents may be administered using the same or different routes of administration in accordance with any medically acceptable method known in the art.

In another embodiment, the pharmaceutical compositions comprising nanoparticles and/or other agents are administered prophylactically, e.g., to prevent infection. Such prophylactic uses will be of particular value for subjects who are immunodeficient, patients who have been treated with immunosuppressive agents, or who have a genetic predisposition or disease (e.g., acquired immunodeficiency syndrome (AIDS), cancer, diabetes, or cystic fibrosis) that makes them prone to developing infections.

In another embodiment, the pharmaceutical compositions comprising nanoparticles and/or antibiotics, and/or other agents are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

Those of ordinary skill in the art will appreciate which conditions the nanoparticles can effectively treat. The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case.

In certain embodiments, multiple therapeutically effective doses of a composition comprising nanoparticles will be administered according to a daily dosing regimen or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, once a week, every other week, and so forth. For example, in some embodiments, a composition comprising nanoparticles will be administered once-weekly, twice-weekly or thrice-weekly for an extended period of time, such as for 1, 2, 3, 4, 5, 6, 7, 8 . . . 10 . . . 15 . . . 24 weeks, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy (i.e., once-weekly, twice-weekly or thrice-weekly administration of a therapeutically effective dose) for one or more weekly cycles until the desired therapeutic response is achieved. The agents can be administered by any acceptable route of administration as noted herein below. The amount administered will depend on the potency of the nanoparticle and its type of functionalization, the magnitude of the effect desired, and the route of administration.

Nanoparticles (again, preferably provided as part of a pharmaceutical preparation) can be administered alone or in combination with one or more other therapeutic agents, such as other agents for treating an infection, including, but not limited to, antibiotics including broad spectrum, bactericidal, or bacteriostatic antibiotics such as penicillins including penicillin G, penicillin V, procaine penicillin, benzathine penicillin, veetids (Pen-Vee-K), piperacillin, pipracil, pfizerpen, temocillin, negaban, ticarcillin, and Ticar; penicillin combinations such as amoxicillin/clavulanate, augmentin, ampicillin/sulbactam, unasyn, piperacillin/tazobactam, zosyn, ticarcillin/clavulanate, and timentin; tetacyclines such as chlortetracycline, doxycycline, demeclocycline, eravacycline, lymecycline, meclocycline, methacycline, minocycline, omadacycline, oxytetracycline, rolitetracycline, sarecycline, tetracycline, and tigecycline; cephalosporins such as cefacetrile (cephacetrile), cefadroxil (cefadroxyl; duricef), cefalexin (cephalexin; keflex), cefaloglycin (cephaloglycin), cefalonium (cephalonium), cefaloridine (cephaloradine), cefalotin (cephalothin; keflin), cefapirin (cephapirin; cefadryl), cefatrizine, cefazaflur, cefazedone, cefazolin (cephazolin; ancef, kefzol), cefradine (cephradine; velosef), cefroxadine, ceftezole, cefaclor (ceclor, distaclor, keflor, raniclor), cefonicid (monocid), cefprozil (cefproxil; cefzil), cefuroxime (zefu, zinnat, zinacef, ceftin, biofuroksym, xorimax), cefuzonam, loracarbef (lorabid) cefbuperazone, cefmetazole (zefazone), cefminox, cefotetan (cefotan), cefoxitin (mefoxin), cefotiam (pansporin), cefcapene, cefdaloxime, cefdinir (sefdin, zinir, omnicef, kefnir), cefditoren, cefetamet, cefixime (fixx, zifi, suprax), cefmenoxime, cefodizime, cefotaxime (claforan), cefovecin (convenia), cefpimizole, cefpodoxime (vantin, pecef, simplicef), cefteram, ceftamere (enshort), ceftibuten (cedax), ceftiofur (naxcel, excenel), ceftiolene, ceftizoxime (cefizox), ceftriaxone (rocephin), cefoperazone (cefobid), ceftazidime (meezat, fortum, fortaz), latamoxef (moxalactam), cefclidine, cefepime (maxipime), cefluprenam, cefoselis, cefozopran, cefpirome (cefrom), cefquinome, flomoxef, ceftobiprole, ceftaroline, ceftolozane, cefaloram, cefaparole, cefcanel, cefedrolor, cefempidone, cefetrizole, cefivitril, cefmatilen, cefmepidium, cefoxazole, cefrotil, cefsumide, ceftioxide, cefuracetime, and nitrocefin; quinolones//fluoroquinolones such as flumequine (Flubactin), oxolinic acid (Uroxin), rosoxacin (Eradacil), cinoxacin (Cinobac), nalidixic acid (NegGam, Wintomylon), piromidic acid (Panacid), pipemidic acid (Dolcol), ciprofloxacin (Zoxan, Ciprobay, Cipro, Ciproxin), fleroxacin (Megalone, Roquinol), lomefloxacin (Maxaquin), nadifloxacin (Acuatim, Nadoxin, Nadixa), norfloxacin (Lexinor, Noroxin, Quinabic, Janacin), ofloxacin (Floxin, Oxaldin, Tarivid), pefloxacin (Peflacine), rufloxacin (Uroflox), enoxacin (Enroxil, Penetrex), balofloxacin (Baloxin), grepafloxacin (Raxar), levofloxacin (Cravit, Levaquin), pazufloxacin (Pasil, Pazucross), sparfloxacin (Zagam), temafloxacin (Omniflox), tosufloxacin (Ozex, Tosacin), clinafloxacin, gatifloxacin (Zigat, Tequin, Zymar-ophthalmic), moxifloxacin (Avelox,Vigamox), sitafloxacin (Gracevit), prulifloxacin (Quisnon), besifloxacin (Besivance), delafloxacin (Baxdela), gemifloxacin (Factive) and trovafloxacin (Trovan), ozenoxacin, danofloxacin (Advocin, Advocid), difloxacin (Dicural, Vetequinon), enrofloxacin (Baytril), ibafloxacin (Ibaflin), marbofloxacin (Marbocyl, Zenequin), orbifloxacin (Orbax, Victas), and sarafloxacin (Floxasol, Saraflox, Sarafin); macrolides such as azithromycin, clarithromycin, erythromycin, fidaxomicin, telithromycin, carbomycin A, josamycin, kitasamycin, midecamycin/midecamycin acetate, oleandomycin, solithromycin, spiramycin, troleandomycin, tylosin/tylocine, roxithromycin, telithromycin, cethromycin, solithromycin, tacrolimus, pimecrolimus, sirolimus, amphotericin B, nystatin, and cruentaren; sulfonamides such as sulfonamide, sulfacetamide, sulfadiazine, sulfadimidine, sulfafurazole (sulfisoxazole), sulfisomidine (sulfaisodimidine), sulfamethoxazole, sulfamoxole, sulfanitran, sulfadimethoxine, sulfamethoxypyridazine, sulfametoxydiazine, sulfadoxine, sulfametopyrazine, and terephtyl; aminoglycosides such as kanamycin A, amikacin, tobramycin, dibekacin, gentamicin, sisomicin, netilmicin, neomycins B, C, neomycin E (paromomycin), streptomycin, plazomicin, amikin, garamycin, kantrex, neo-fradin, netromycin, nebcin, humatin, spectinomycin(Bs), and trobicin; carbapenems such as imipenem, meropenem, ertapenem, doripenem, panipenem/betamipron, biapenem, tebipenem, razupenem (PZ-601), lenapenem, tomopenem, and thienamycin (thienpenem); ansamycins such as geldanamycin, herbimycin, rifaximin, and xifaxan; carbacephems such as loracarbef and lorabid; carbapenems such as ertapenem, invanz, doripenem, doribax, imipenem/cilastatin, primaxin, meropenem, and merrem; glycopeptides such as teicoplanin, targocid, vancomycin, vancocin, telavancin, vibativ, dalbavancin, dalvance, oritavancin, and orbactiv; lincosamides such as clindamycin, cleocin, lincomycin, and lincocin; lipopeptides such as daptomycin and cubicin; macrolides such as azithromycin, zithromax, surnamed, xithrone, clarithromycin, biaxin, dirithromycin, dynabac, erythromycin, erythocin, erythroped, roxithromycin, troleandomycin, tao, telithromycin, ketek, spiramycin, and rovamycine; monobactams such as aztreonam and azactam; nitrofurans such as furazolidone, furoxone, nitrofurantoin, macrodantin, and macrobid; oxazolidinones such as linezolid, zyvox, vrsa, posizolid, radezolid, and torezolid; polypeptides such as bacitracin, colistin, coly-mycin-S, and polymyxin B; drugs against mycobacteria such as clofazimine, lamprene, dapsone, avlosulfon, capreomycin, capastat, cycloserine, seromycin, ethambutol, myambutol, ethionamide, trecator, isoniazid, I.N.H., pyrazinamide, aldinamide, rifampicin, rifadin, rimactane, rifabutin, mycobutin, rifapentine, priftin, and streptomycin; and other antibiotics such as arsphenamine, salvarsan, chloramphenicol, chloromycetin, fosfomycin, monurol, monuril, fusidic acid, fucidin, metronidazole, flagyl, mupirocin, bactroban, platensimycin, quinupristin/dalfopristin, synercid, thiamphenicol, tigecycline, tigacyl, tinidazole, tindamax fasigyn, trimethoprim, proloprim, and trimpex; adjuvants, including aluminum salts (alum), such as aluminum hydroxide, aluminum phosphate, aluminum sulfate, etc.; oil-in-water emulsion formulations; (saponin adjuvants; Complete Freund's Adjuvant (CFA) and Incomplete Freund's Adjuvant (IFA); cytokines, such as interleukins (IL-1, IL-2, IL-4, IL-5, IL-6, IL-7, IL-12, interferons, macrophage colony stimulating factor (M-CSF), tumor necrosis factor (TNF), etc.; detoxified mutants of a bacterial ADP-ribosylating toxin such as a cholera toxin (CT), pertussis toxin (PT), or an E. coli heat-labile toxin (LT); oligonucleotides comprising CpG motifs; as well as other immunostimulatory molecules; and vaccines such as vaccines against tuberculosis, diphtheria, tetanus, pertussis, Haemophilus influenzae type B, cholera, typhoid, and Streptococcus pneumoniae, and other vaccines comprising bacterial antigenic proteins or attenuated or dead bacteria for boosting an immune response against bacteria, or other medications used to treat a particular condition or disease according to a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day.

Nanoparticles can be administered prior to, concurrent with, or subsequent to other agents. If provided at the same time as other agents, nanoparticles can be provided in the same or in a different composition. Thus, nanoparticles and one or more other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising nanoparticles and a dose of a pharmaceutical composition comprising at least one other agent, such as another drug for treating an infection, which in combination comprise a therapeutically effective dose, according to a particular dosing regimen. Similarly, nanoparticles and one or more other therapeutic agents can be administered in at least one therapeutic dose. Administration of the separate pharmaceutical compositions can be performed simultaneously or at different times (i.e., sequentially, in either order, on the same day, or on different days), as long as the therapeutic effect of the combination of these substances is caused in the subject undergoing therapy.

### Kits

Kits may comprise one or more containers of the compositions described herein comprising functionalized nanoparticles, or reagents for preparing such compositions, and optionally one or more antibiotics for treating a bacterial infection. Compositions can be in liquid form or can be lyophilized. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle). The kit can further comprise a container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device. The kit may also provide a delivery device pre-filled with the functionalized nanoparticles.

In addition to the above components, the subject kits may further include (in certain embodiments) instructions for practicing the subject methods (i.e., instructions for treating a bacterial infection with nanoparticles as described herein). These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, Blu-ray, flash drive, and the like, on which the information has been recorded. Yet another form of these instructions that may be present is a website address which may be used via the internet to access the information at a removed site.

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

Engineered Gold Nanoclusters Overcome the Drug-Refractory State Associated with Biofilm Formation

The challenge of targeting persister cells is highlighted by their differing response to cell injury and improved repair mechanisms. Bactericidal antibiotics commonly induce reactive oxygen species (ROS), especially hydroxyl radicals (HO.), leading to cell death^{12, 13}. Persister cells, however, respond to ROS stress by not producing HO′ radicals. Instead superoxide dismutase (SOD) activity converts the superoxide radical (O₂.⁻) into hydrogen peroxide (H₂O₂), which is finally converted to water (H₂O) by catalase¹⁴. If antibiotics bypass this first line of defense, DNA repair mechanisms allow persister cells to survive¹⁵. We hypothesized that a material capable of effectively blocking the repair of damaged DNA in persister cells and, at the same time, increasing HO. radical production from the catalytic decomposition of H₂O₂ would restore or potentiate the activity of conventional antibiotics against P. aeruginosa biofilm-associated persister cells.

Here we demonstrated the ability of a novel engineered gold nanocluster (AuNC@CPP) to treat an in vitro biofilm of a clinical isolate of P. aeruginosa and in vivo, using a validated model of P. aeruginosa biofilm chronic disease, in comparison to a standard of care antibiotic, 3000 μg/mL of FLOXIN®Otic. Successfully targeting persister cells using this method will increase the ability to eradicate many chronic biofilm infectious diseases, recover the resistance profiles of commonly used antibiotics and reduce the potential for development of antimicrobial resistance.

Ofloxacin Fails to Eradicate In Vitro Biofilm from Otopathogenic P. aeruginosa.

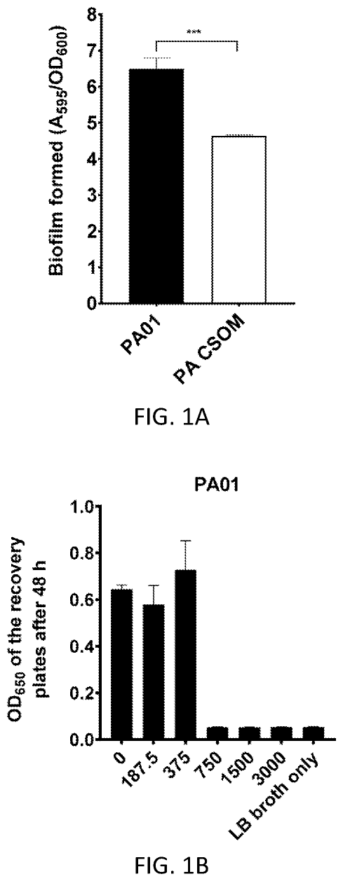

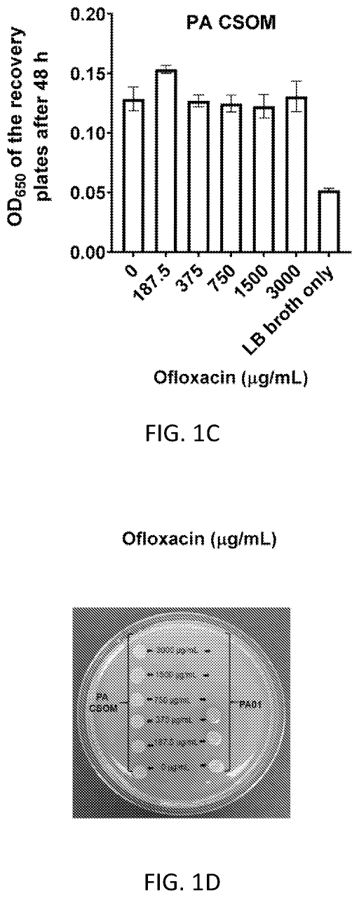

Delivering high antibiotic concentrations through topical administration is a therapeutic strategy proposed to treat problematic bacterial pathogens in a biofilm¹⁶. To challenge this hypothesis, we used two strains of P. aeruginosa, including antibiotic sensitive wild-type reference strain PA01 and a clinical otopathogenic strain isolated from CSOM patient (PA CSOM). Biofilms were challenged with a fluoroquinolone as they are commonly used to treat infections caused by P. aeruginosa and the current standard of care in CSOM¹⁷⁻¹⁹, the only available antibiotic effective at killing both growing and non-growing cells²⁰, and they have no restriction to diffuse into P. aeruginosa biofilms²¹. To demonstrate that the diffusion barrier of the bacterial biofilm (dense cell cluster and sticky biofilm matrix) plays a minor role in defense against fluoroquinolone attacks, we first compared the biofilm formation capacity of PA CSOM and PA01. We quantified the biofilm formation capacity by the normalized biofilm matrix value (crystal violet stained biofilm absorbance at 595 nm, A595/total cell growth, optical density at 600 nm, OD₆₀₀). We found that PA01 is the highest biofilm producer (FIG. 1A). The minimum inhibitory concentration (MIC) of ofloxacin against PA01 and PA CSOM were between 1.6-3.3 μg/mL and 52.5-100 μg/mL, respectively. In vitro resistance of P. aeruginosa to ofloxacin is defined as MIC>8 μg/mL (Clinical microbiology reviews. 2012, 25, 545-582), suggesting that PA CSOM is a fluoroquinolone-resistant strain.

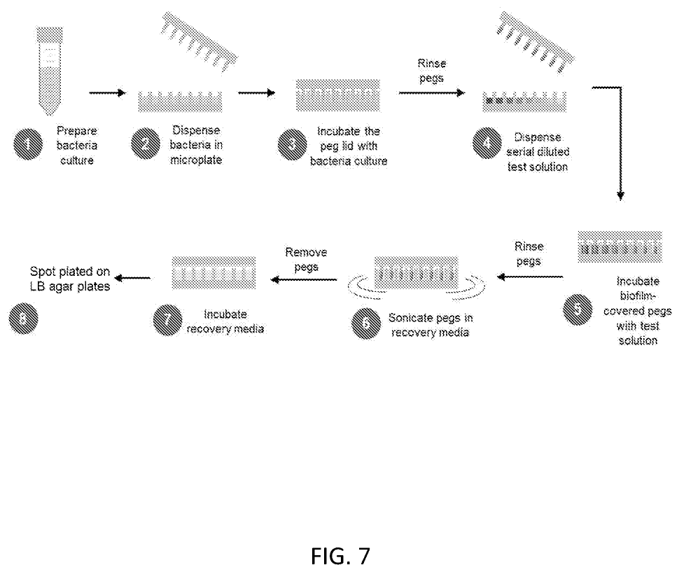

Then, the minimum biofilm eradication concentration (MBEC) of FLOXIN®Otic against 48 h old PA01 and PA CSOM biofilms were determined using the commercially available MBEC Assay® (Innovotech Inc. Edmonton, Canada). This new technology is useful for predicting clinical failure and clinical success of antimicrobial therapy against biofilm bacteria^{22,23} In this assay, the MBEC is identified when incubated recovery media has an optical density at 650 nm (OD₆₅₀)<0.1 or no regrowth of bacteria when spotted on Luria broth (LB) agar plates (FIG. 7). Our results show that ofloxacin has a MBEC of 750 μg/mL (OD₆₅₀ 0.1) against 48 h old PA01 biofilm (FIG. 1B). This finding is consistent with that of Masadeh who reported a MBEC of 640 μg/mL against 24 h old strain P. aeruginosa (ATCC 27853) biofilm²⁴. Contrary to the results for PA01, no eradication of the 48 h old PA CSOM biofilm was obtained after treatment with 3000 μg/mL of ofloxacin, the concentration in the commercial preparation (FLOXIN®Otic). The incubated recovery media from all concentrations tested showed an OD₆₅₀>0.1 (FIG. 1C). To confirm the MBEC of ofloxacin against 48 h old PA01 and PA CSOM biofilms, the recovery media were spotted on Luria broth (LB) agar plates and incubated for 48 h at 37° C. As shown in the photograph (FIG. 1D), the treatment of 48 h old PA01 biofilm with ofloxacin at 750 μg/mL left no viable cells compared to bacterial growth after recovery of 48 h old PA CSOM treated with ofloxacin at 3000 μg/mL. Although PA CSOM produced quantitatively less biofilms than PAO1, it had the highest tolerance to ofloxacin. Given that PAO1 produced more biofilm that PA CSOM, yet had a much higher susceptibility to ofloxacin, we concluded that the diffusion barrier of the biofilm plays a minor role in the defense against fluoroquinolones. We hypothesized that the fraction of persister cells is the key parameter that regulates the tolerance to fluoroquinolones, as we investigated later. Of note, the maximum concentration of ofloxacin in otorrhea of CSOM patients with a persistent purulent discharge ranged from 388.8 to 2849.8 μg/mL at 30 minutes after topical administration of FLOXIN®Otic²⁵. The high local levels of ofloxacin (≥4×MIC of PA CSOM) will kill all of the fluoroquinolone-resistant P. aeruginosa population. The in vitro bactericidal activity of ofloxacin against PA CSOM biofilms suggested that FLOXIN®Otic would not eradicate persister cells from a draining ear infected with this clinical otopathogenic strain of P. aeruginosa, leaving the patients at continuing risk of recurrence. Our results highlight that delivering a high concentration of ofloxacin by topical administration is not an effective strategy for treating problematic bacterial biofilms.

### Increasing the Fraction of Persister Cells Promotes Biofilm Tolerance to Ofloxacin

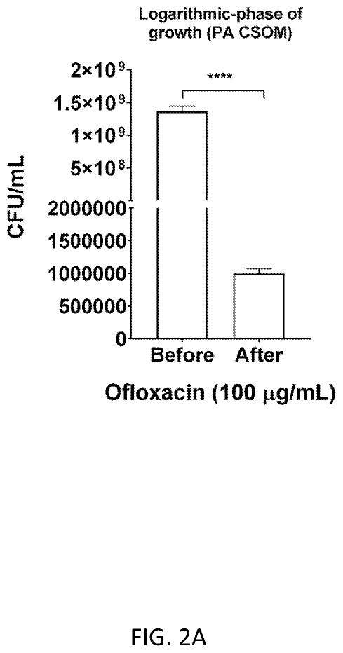

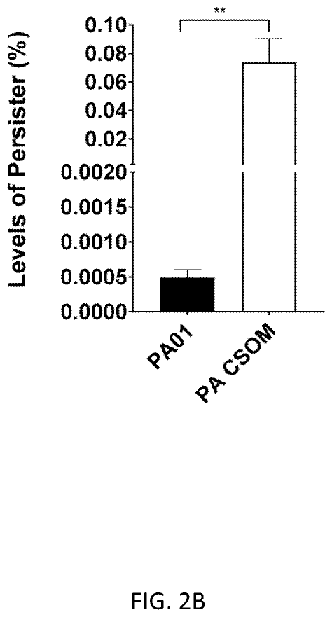

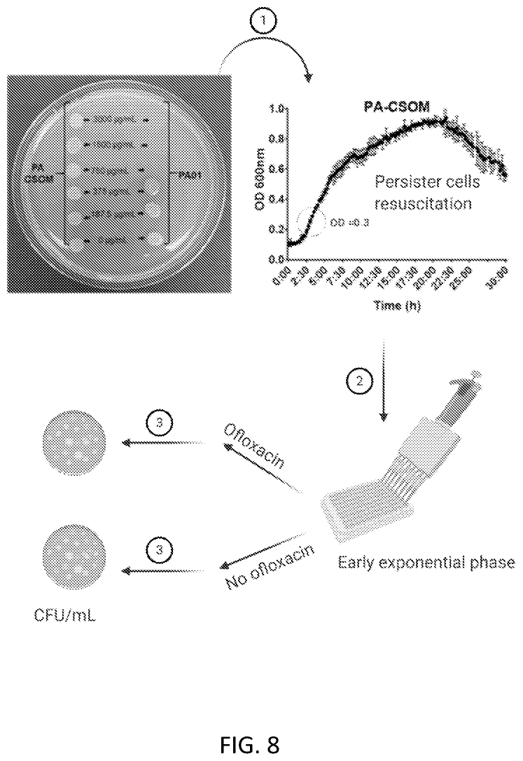

When ofloxacin is used at 100 μg/mL, the survival of the P. aeruginosa biofilm does not depend on drug resistance mechanisms such as the presence of efflux pumps²⁶. To show that PA CSOM was not resistant to ofloxacin when used at 3000 μg/mL, the planktonic logarithmic-phase culture of surviving persister cells was challenged with 100 μg/mL of ofloxacin (FIG. 8). We found that the number of bacteria per milliliter (CFU/mL) decreased by 3 log 10 after the ofloxacin treatment (FIG. 2). This result suggests that when growth resumes, the persister cells of PA CSOM give rise to viable progeny that are sensitive to ofloxacin. The drug-refractory state associated with PA CSOM biofilm formation is mediated by tolerance rather than resistance to the antibiotic.

To confirm our hypothesis that the high fraction of persister cells is the main reason why 3000 μg/mL of ofloxacin fails to eradicate the PA CSOM biofilm, we compared the persister cell formation capacity of PA CSOM and PA01. We found that the logarithmic culture of PA01 produced a tiny fraction of persister cells equal to 0.0005% of total bacteria population (FIG. 2), which is similar to that previously reported²⁷. In comparison, the logarithmic culture of PA CSOM produced a large fraction of persister cells equal to 0.07% (140-fold increase compared to PA01) (FIG. 2). The increased fraction of persister cells in the clinical isolate promotes tolerance to ofloxacin.

It has been shown that P. aeruginosa strains producing high levels of persister cells (termed high-persister mutants) are selected for in course of antibiotic treatment in cystic fibrosis patients²⁸. Given that CSOM is commonly treated by multiple courses of topical fluoroquinolone treatment, we conclude that PA CSOM, evaluated by the same parameters, is likely a high-persister mutant selected by the same process.

AuNC@CPP Overcome the Drug-Refractory State Associated with Biofilm Formation

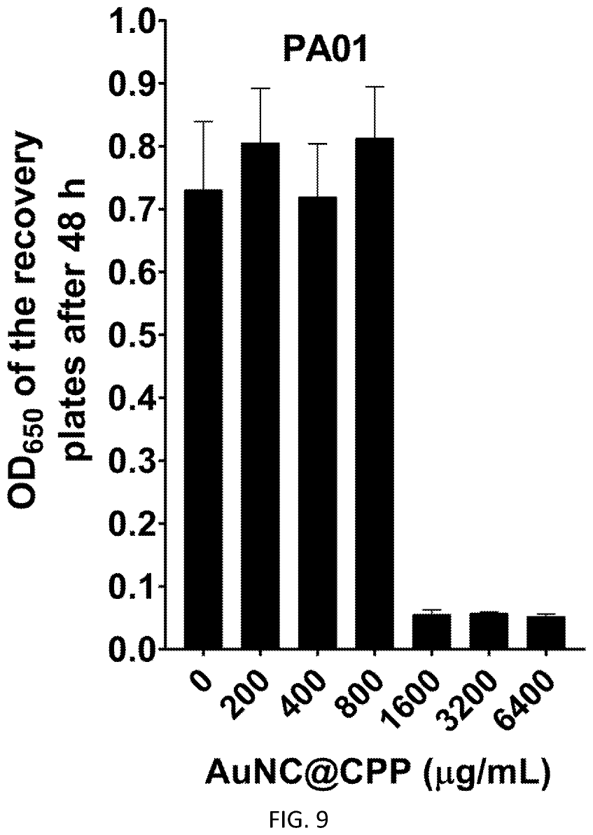

As demonstrated above, 48 h old PA CSOM biofilm was tolerant to 3000 μg/mL of ofloxacin, the concentration in the available commercial preparation (FLOXIN®Otic). This highlights the therapeutic challenge of achieving adequate concentration in the middle ear mucosae in CSOM patients²⁹ where mucosal FLOXIN®Otic concentrations range from nondetectable to 602 μg/mL after topical administration²⁵. Consequently, we needed to design an approach to develop a nanomaterial capable of lowering the MBEC of ofloxacin into a range achievable clinically. We engineered a gold nanocluster (AuNC@CPP) that comprises a cell-penetrating peptide (CPP) (YGRKKRRQRRR SEQ ID NO:7)-(β-ala)₃-Cys-amide and thiolated polyethylene glycol with a carboxyl termination (an efficient protecting ligand that confers good stability to AuNC@CPP in solution as well as in biological systems). The UV-Vis spectrum of AuNC@CPP showed a monotonous decrease from UV into the visible but no surface plasmon resonance peak at 520 nm indicative of the formation of ultrasmall particles (core diameter≤2 nm)³⁰ (FIG. 9A).

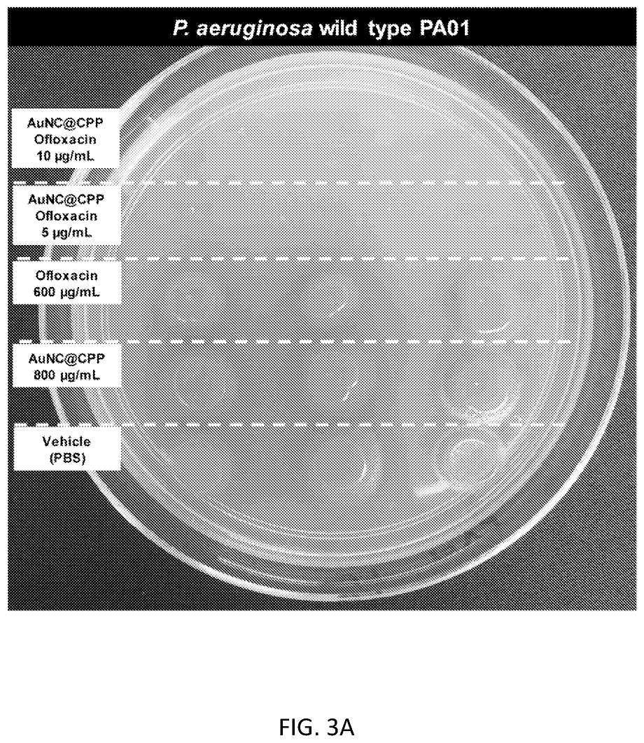

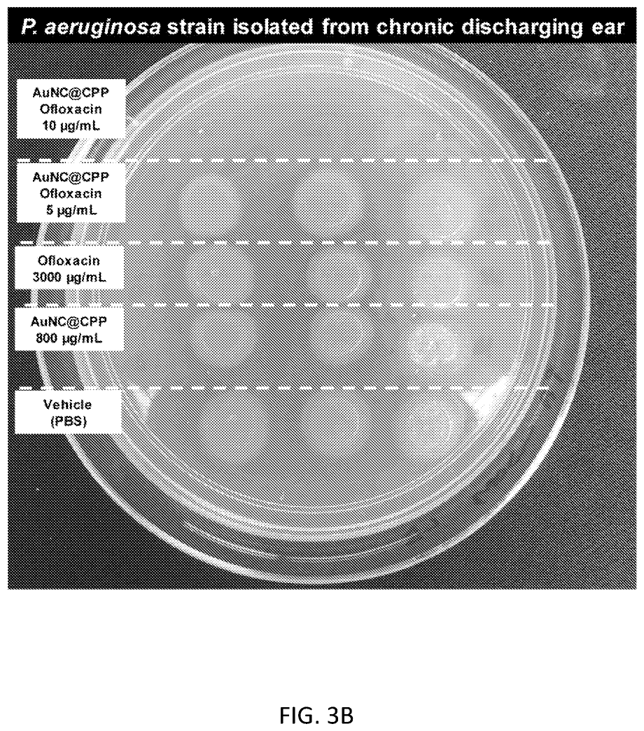

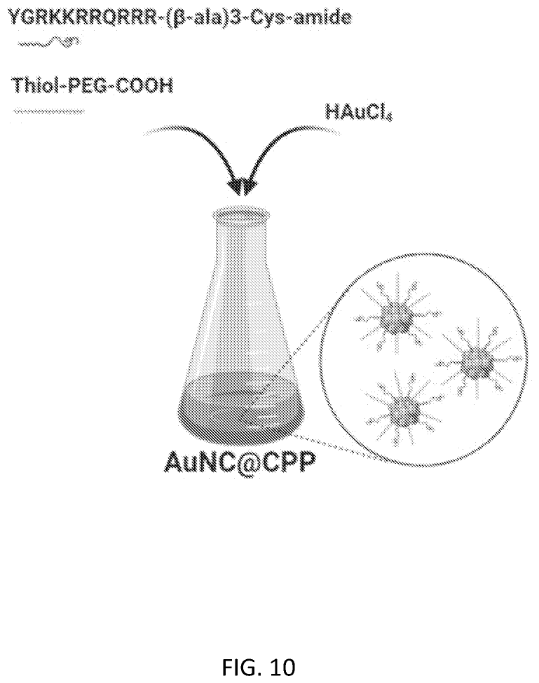

The minimum bacteriocidal concentration (MBC), defined as the lowest concentration that resulted in no bacterial growth following removal of the drug, of ofloxacin has been reported as 5 μg/mL against the sensitive strain P. aeruginosa (ATCC 27853)²⁴. Of note, AuNC@CPP alone exhibits a MBEC of 1600 μg/mL (OD₆₅₀≤0.1) against 48 h old PA01 biofilm (FIG. 10). We demonstrated that AuNC@CPP lowers the MBEC of ofloxacin against 48 h old PA01 and PA CSOM biofilms to concentrations equal to 1×MBC and 2×MBC of ofloxacin, respectively against P. aeruginosa ATCC 27853. As shown in the photograph (FIG. 3), the treatment of 48 h old PA CSOM biofilm with AuNC@CPP (800 μg/mL) alone, ofloxacin at 3000 μg/mL and combination of AuNC@CPP plus ofloxacin at 5 μg/mL did not eradicate the biofilm and associated persister cells. Remarkably, an AuNC@CPP and 10 μg/mL of ofloxacin combination leaves no viable cells (>300-fold reduction in MBEC). Similarly, the treatment of 48 h old PA01 biofilm with AuNC@CPP plus ofloxacin at 5 μg/mL leaves no viable cells (150-fold reduction in MBEC). Together, these findings highlight that AuNC@CPP restores or potentiates the activity of ofloxacin against the P. aeruginosa biofilm and associated persistent cells, showing that there is much to be gained, even with existing drugs, by taking advantage of synthetic nanotechnology.

### Biocompatibility Assessments

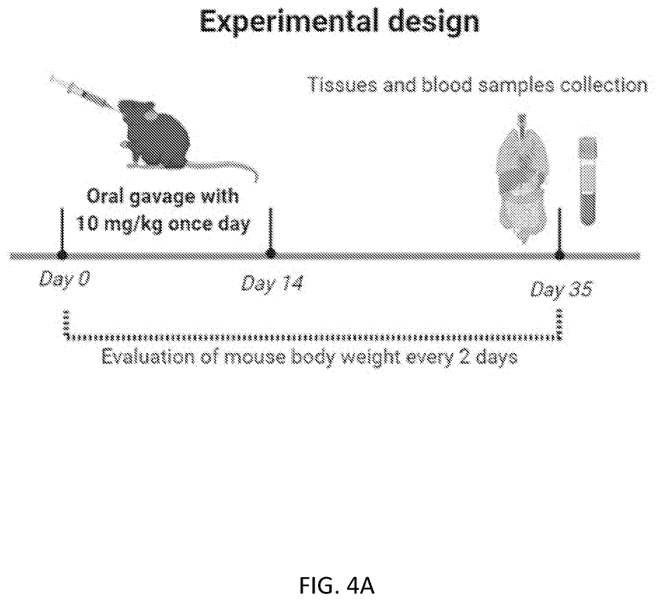

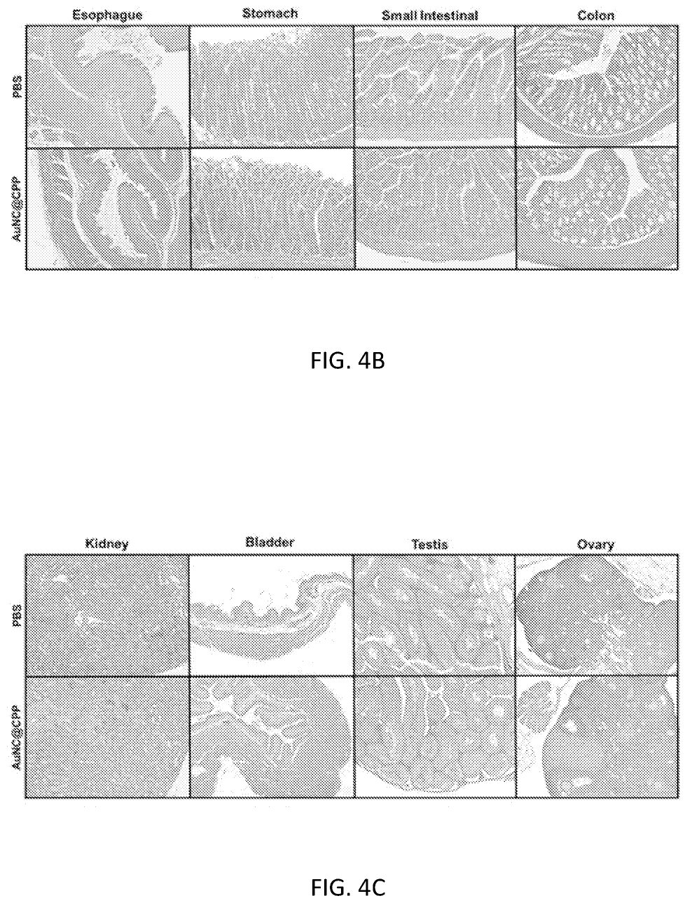

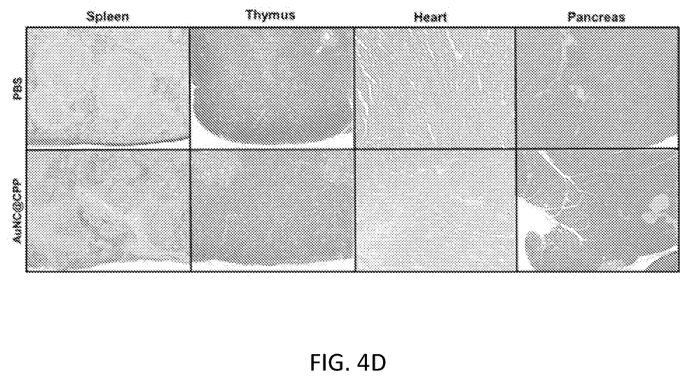

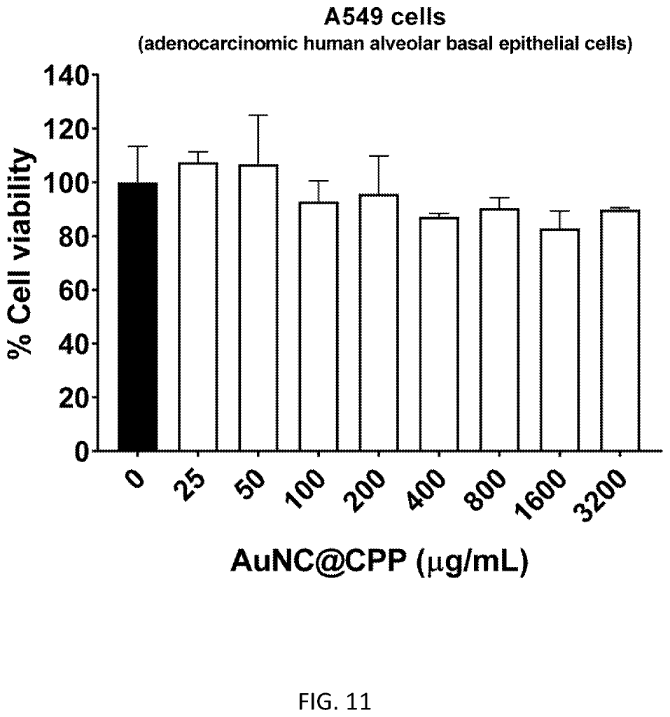

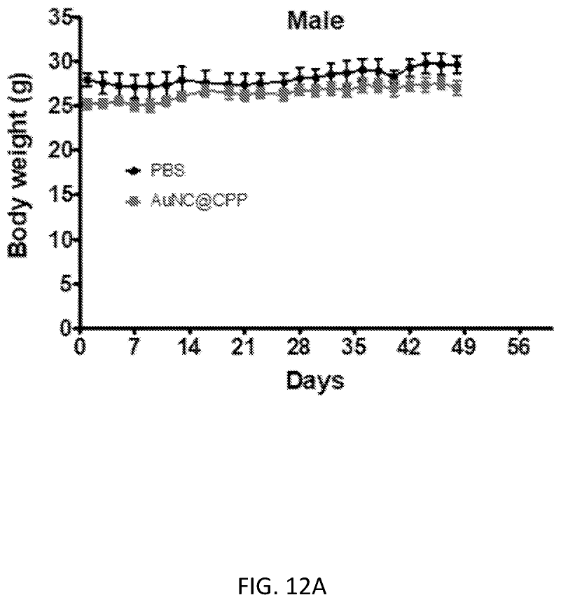

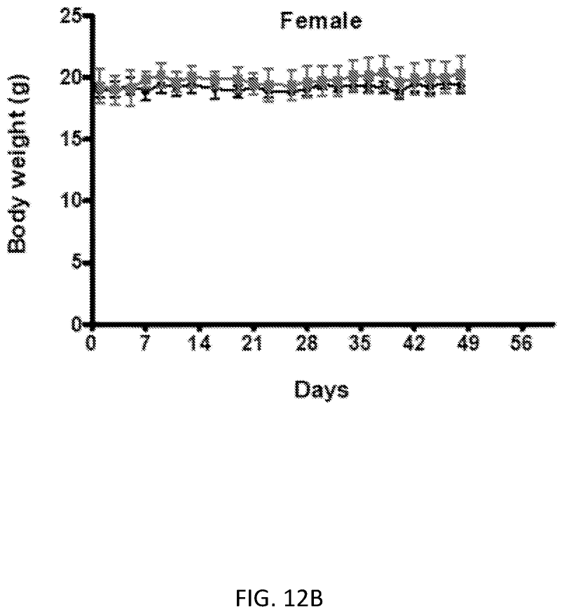

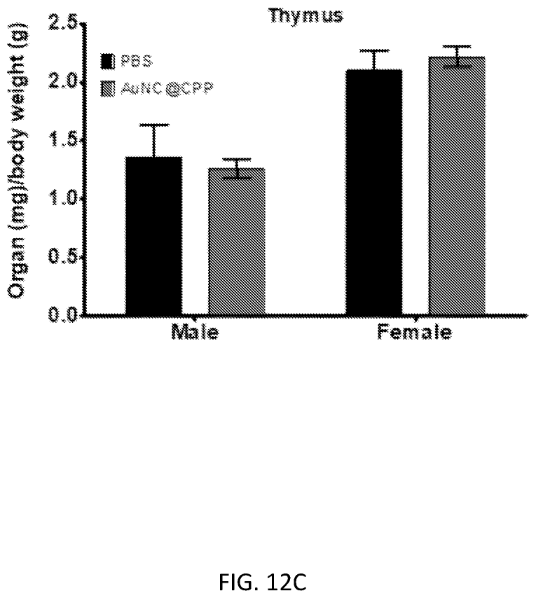

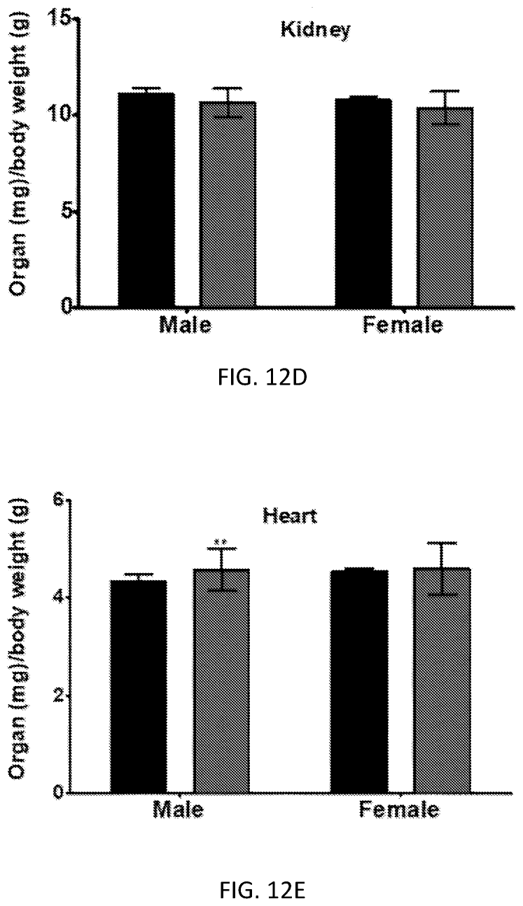

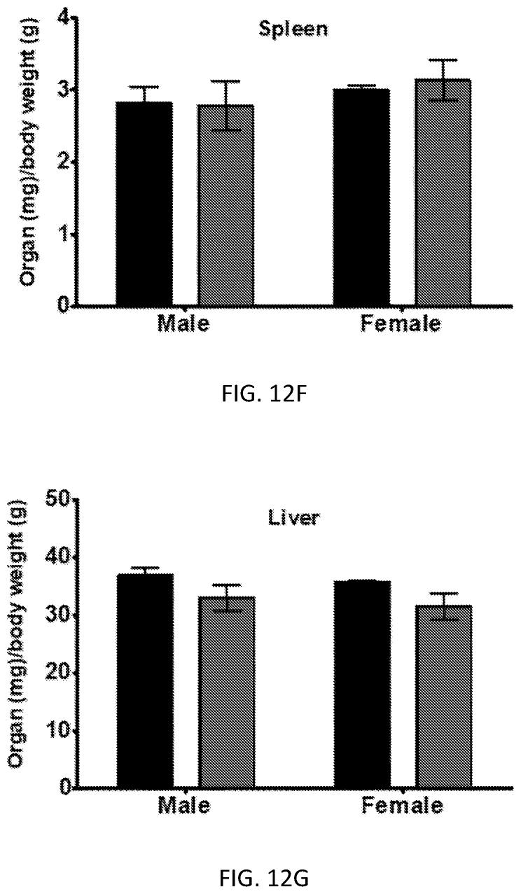

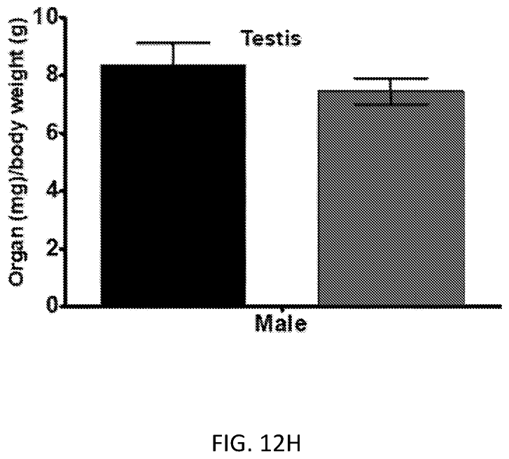

Prior to moving in vivo, cytotoxicity is an important factor in assessing the potential for adverse effects of a therapeutic compound in vivo. We used an industry standard MTT viability assay to test the cytotoxicity of AuNC@CPP against adenocarcinomic human alveolar basal epithelial cells (A549 cells). We found that cells exhibited more than 90% viability upon direct exposure to AuNC@CPP at 3200 μg/mL (FIG. 11). Furthermore, oral administration of AuNC@CPP is unlikely to be of concern for systemic toxicity or in the induction of gastrointestinal illnesses as shown in healthy mice up to 35 days after administration by oral gavage at a dose of 10 mg/kg (or 1000 μg/mL) daily for 14 days. No statistical significance in body weight loss was seen between PBS (control) and AuNC@CPP or between sexes (FIG. 12). In addition, there was no noticeable change in fecal form during the observations. AuNC@CPP does not prompt significant change in hematologic or liver and kidney function (Tables 2 and 3). There was no change in almost all organ-to-body weight ratios (FIGS. 4C-4H). The heart-to-body weight ratio was significantly increased in the male treated AuNC@CPP group (FIG. 4E). We did not consider this to be a toxicity because it does not corroborate the histopathological data³¹.

Light microscopic examination of sections of organs of PBS (control) and treated AuNC@CPP group showed a normal histology and absence of any gross pathological lesions (FIG. 4). Further studies of increasing time exposure and doses are necessary to determine if toxicity occurs at higher doses or if there is a no adverse effects limit.

### Antipseudomonal Therapy Floxin®Otic Plus AuNC@CPP is Superior to Floxin®Otic Alone in Mouse Model of CSOM

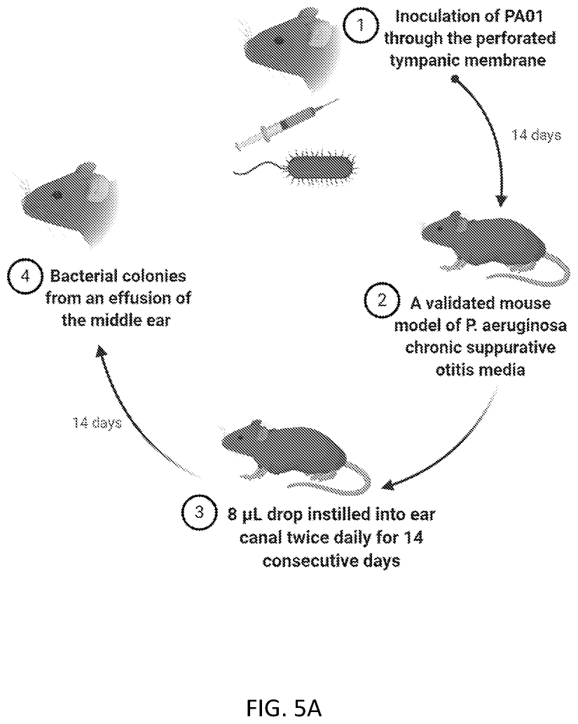

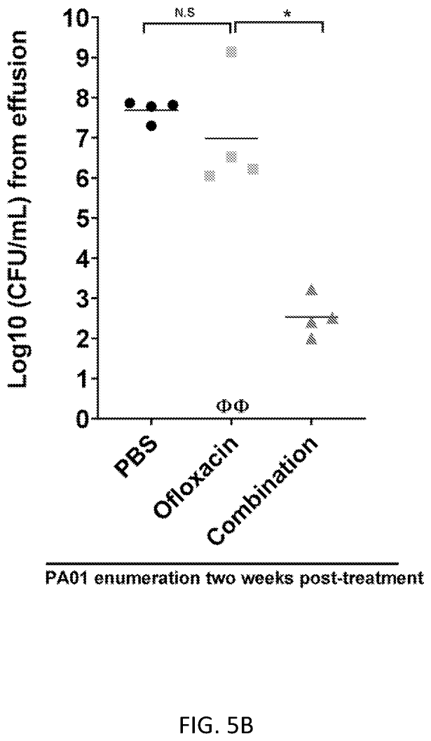

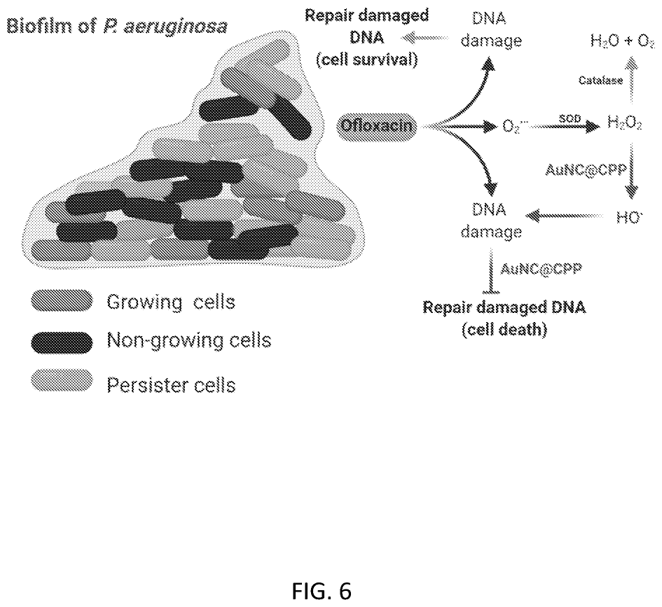

To determine the clinical relevance of this therapy adjuvant, the in vivo efficacy of Floxin®Otic was compared with that of Floxin®Otic plus AuNC@CPP. We have chosen CSOM as an in vivo model of P aeruginosa biofilm-related infections given its significant global burden as the leading cause of hearing loss in children in developing countries, with an incidence of 31 million cases and prevalence of >300 million^{32, 33, 34}. CSOM currently has no cure with end stage disease ending in surgery, not available where resources are often limited or non-existent³⁵. We recently developed and validated a mouse model of P. aeruginosa CSOM to test potential therapeutics. The primary endpoint of our in vivo study was the level of bacterial colonies from effusion of the middle ear 14 days after stopping treatment. The treatment regime was chosen to mimic that which is already prescribed in the clinic. Treatments were commenced 14 days after inoculations were performed to create CSOM. An 8 μL drop was placed in the ear canal twice a day for 14 consecutive days. Since AuNC@CPP concentration used for the in vivo study is 5.4 times lower than the MBEC (1600 μg/mL) against PA01 biofilm, we performed a superiority study with three arms, including the placebo control group (phosphate-buffered saline, PBS), FLOXIN®Otic (24 μg of ofloxacin) and combination (24 μg of ofloxacin+296 μg of AuNC@CPP). Administration of FLOXIN®Otic decreased the viable counts in middle ear effusion 14 days after stopping treatment (FIG. 5 and Table 1). However, no notable difference was observed between mice treated with FLOXIN®Otic and placebo control (PBS) in this model, suggesting that the current standard of care did not maintain clinical benefit once treatment is stopped. A combination of FLOXIN®Otic and AuNC@CPP led to a significant decrease (2.54±0.51 vs 7.69±0.26 Log 10 CFU/mL and p<0.05) in viable accounts compared to FLOXIN®Otic alone, a benefit of over 5 log reduction, (FIG. 5 and Table 1). This result illustrates the exciting opportunities that nanotechnology offers for overcoming the drug-refractory state associated with biofilm formation. In a nutshell, these findings show that AuNC@CPP plus fluoroquinolone combination could lead to new ways of effectively treating chronic biofilm infections. Further research is planned to determine optimal dose and schedule for maximum in vivo efficacy.

### Mechanism of AuNC@CPP Action

The bacterial cells embedded in the P aeruginosa biofilm produce highly acidic niches with pH values close to 4.5. Our data demonstrate that AuNC@CPP exhibits peroxidase-like activity at acidic pH 4.5 that catalyzes decomposition of H₂O₂ into HO resulting in oxidization of 3,3′,5,5′ tetramethylbenzidine (TMB) to develop a blue color solution (FIG. 6A). The bacterial response to DNA damage (known as the SOS response) occurs in three stages: (i) RecA protein activation; (ii) the autocatalytic cleavage of the LexA repressor; and (iii) the derepression of SOS genes needed for the repair of DNA³⁶. To show that AuNC@CPP blocks DNA repair, we first evaluated the ability of AuNC@CPP to inhibit RecA protein activation. Monitoring ATPase activity can be used as a method for in vitro screening of RecA inhibitory drugs^{37, 38}. AuNC@CPP is capable of inhibiting the RecA ATP hydrolysis activity.

### Discussion

Disruption or dispersal is a commonly pursued treatment strategy for biofilm diseases³³⁻⁴¹. Biofilm dispersal could have negative consequences in vivo. Any strategy needs to be carefully coordinated with an active agent capable of targeting the dispersed cells. Dispersed biofilm cells represent a distinct stage in the transition from biofilm to planktonic lifestyles and are highly virulent against macrophages compared with planktonic, non-biofilm cells⁴². A reminder of the clinical risk in this approach is that in vivo dispersion of mobile biofilm bacteria has been shown to cause fatal sepsis in the absence of antibiotic therapy in a mouse wound model⁴³. Furthermore, if the persister cells, which retain their phenotype for days or weeks after withdrawal from biofilm, are not directly addressed the chronic infection cycle will continue^{44, 45}. Therefore, the clinical use approaches that involve the dispersal of biofilms to potentiate killing by antibiotics may increase the risk of an uncontrolled infection and expose patients to serious sequealae.

We demonstrate that AuNC@CPP is a potential solution for this treatment challenge. AuNC@CPP leverages a peroxidase-like activity to enhance HO. production through the catalytic decomposition of H₂O₂ produced in persister cells by SOD activity. Besides increasing the production of HO radicals, AuNC@CPP also blocks the DNA repair through the inhibition of RecA ATPase activity and LexA cleavage activity of RecA. Therefore, AuNC@CPP could be widely beneficial to synergistic DNA-damaging agents to target bacteria that require the SOS response for survival, a general principle of killing that may be applicable to other bacteria. It remains to be defined which FDA-approved fluoroquinolone (including moxifloxacin, ciprofloxacin, gemifloxacin, levofloxacin, ofloxacin and finafloxacin) would serve as the best to coadminister along with AuNC@CPP.

Another aspect of the current antibiotic crisis is drug resistance characterized by a higher minimum inhibitory concentration (MIC). Recently, RecA has been validated as a key target for to lower the MIC of multiple antibiotics⁴⁶. For example, E. coli strain deficient in RecA exhibits an increased sensitivity (4 to 32-fold reduction in MIC) to mitomycin C, levofloxacin, and novobiocin relative to the wild type⁴⁶. Moreover, RecA knockout strain showed greater reductions in mutation rate to multiple antibiotics relative to the wild type⁴⁶. Since AuNC@CPP is a RecA inhibitor, it has the potential to be exploited to lower the MIC of antibiotics against resistant bacteria or could plausibly slow acquired drug resistance to multiple antibiotics. In this way, using AuNC@CPP has potential as a first line adjuvant in the approach to antimicrobial resistance.

### Methods

### Synthesis of Cell Penetrating Peptide (CPP)

Ac-(YGRKKRRQRRR SEQ ID NO:7)-(β-Ala)-(β-Ala)-(β-Ala)-Cys-CONH₂ (CPP) was synthesized using a ABI 433A automatic peptide synthesizer on a 0.25 mmol scale by standard fluorenylmethyloxycarbonyl (Fmoc) solid-phase peptide synthesis (SPPS) on a Novabiochem NovaPEG Rink Amide resin. The reactions were carried out utilizing 4-fold excess of Fmoc-amino acids catalyzed with 1 eq of HTBU and Oxyma Pure and 2 eq. of DIEA. The coupling time was 1 h. After all amino acid residues were coupled mechanically, an acetyl group was coupled manually onto the N-terminus by adding Oxyma Pure and acetic anhydride in N, N-dimethylformamide (DMF) and shook for 1 h at room temperature. Peptidyl resin was washed with N, N-dimethylformanide (DMF) and dichloromethane (DCM) and then dried. The peptidyl resin was treated with trifluoroacetic acid (TFA)/Phenol/triisopropylsilane (TIS)/water (92.5:2.5:2.5:2.5) for 3 h. The cleavage cocktail solution was removed by evaporation, and the peptide was precipitated by cold diethyl ether. The peptide was purified by reverse-phase high performance liquid chromatography (RP-HPLC) using Waters system. The molecular masses were determined by MALDI-TOF using the Voyager-DE RP Biospectrometry Workstation instrument.

### Synthesis of AuNC@CPP

Freshly prepared aqueous solutions of HAuCl₄ (20 mM, 1 mL) and thiol thio-carboxy poly(ethylene glycol) (PEG-COOH, 18 mg, 2 mL) and CPP (18 mg, 2 mL) were mixed in water (13.4 mL). After that, an aqueous NaOH solution (1 M, 1.2 mL) was added to the mixture. A freshly prepared NaBH₄ solution (112 mM) was obtained by dissolving 43 mg of NaBH₄ in 2 mL of NaOH solution (1 M), followed by the addition of 8 mL of ultrapure water. After that, 0.4 mL of NaBH₄ solution was added into the solution, and the AuNC@CPP were collected after 24 h. After synthesis, the solutions were dialyzed (dialysis membrane, MWCO=1000) for 2 days against Milli-Q water, which was changed every 8 h to remove the unconjugated thiol PEG-COOH and CPP. The resulting AuNC@CPP were lyophilized and dried completely before further use. The UV-vis (ultraviolet-visible) absorption measurements were carried out using an absorption spectrophotometer (spectramMax M2, Molecular Devices, Downington, Pa.).

### Bacterial Strains, Growth Media, and Conditions

We used two strains of P. aeruginosa, including antibiotic sensitive wild-type reference strain PA01 and a clinical otopathogenic strain isolated from CSOM patient (PA CSOM). In all experiments, bacterial cells were cultured in 10 mL of Luria-Bertani (LB) broth at 37° C. and were aerated at 225 p.r.m in 50 mL tube plastic (polypropylene). Exponential phase cultures were prepared as follows: a stationary overnight culture was diluted 1:1,000 in LB and incubated at 37° C. with aeration at 225 r.p.m. until optical density at 600 nm (OD₆₀₀)=0.3 was reached.

### Quantification of Biofilm Formation

Biofilms were prepared as follows: a stationary overnight cultures of each PA01 and PA CSOM was inoculated into wells of 96-well microtiter plates containing 150 μl LB medium. Inoculated plates were incubated at 37° C. without shaking for 24 h. After incubation, the growth was confirmed as the optical density at 600 nm (OD₆₀₀) using a microplate reader (spectramMax M2, Molecular Devices, Downington, Pa.), after which the LB was removed from each well and the wells rinsed with phosphate buffered saline (PBS) to remove planktonic cells. Crystal violet (CV) solution (0.1%, w/v) of was applied and the microtiter plate was incubated at room temperature for 10 min followed by PBS washing to remove the excess CV. 10% acetic acid was added to each well to extract the CV stain. Absorbance of CV staining was read at 595 nm (A₅₉₅) using the microplate reader. Results were analyzed as the ratio of the CV staining (biofilm matrix, A₅₉₅) to the total cell growth (OD₆₀₀).

### Persistence Assays

PA01 and PA CSOM cultures were incubated at 37° C. with shaking. 1 mL of culture from each strain was aliquoted when reaching an OD₆₀₀ of 0.3. Cultures were gently rinsed to remove LB medium and replaced with 1 mL of PBS. Ofloxacin (100 μg/mL) was added to the culture flasks (except for the control flasks). After 24 h incubation, ofloxacin was removed and the number of colony-forming units per milliliter (CFU/mL) was determined. It is important to incubate the bacterial cells for 48 h to ensure that all of the persister cells are re-growing. The fraction of persister cells was calculated as the ratio of the CFU/mL after ofloxacin treatment to the CFU/mL of the control flasks.

### Biofilm Eradication Assay

Overnight culture of either PA01 or PA CSOM was diluted 1:1,000 in fresh LB medium and add 150 μL of the dilution per well in a MBEC Assay®Biofilm Inoculator with 96 well. Biofilms were allowed to develop into Pegs lig for 48 h without shaking. The pegs lid were gently rinsed to removed planktonic bacteria and incubated in a new MBEC Assay®Biofilm Inoculator with 96 well containing a serial dilution of tested solution in PBS. These MBEC Assay®Biofilm Inoculator with 96 well were incubated for 24 h. Then the Pegs lig were washed and sonicated for 15 min in a new MBEC Assay®Biofilm Inoculator with 96 well containing fresh medium (recovery media). Following incubation for 48 h, the optical density at 650 nm (OD₆₅₀) was read using a microplate reader (spectramMax M2, Molecular Devices, Downington, Pa.). Wells with an OD650 of less than 0.1 is evidence of biofilm eradication. The MBEC value was defined as the lowest concentration of drug that eradicate the biofilm (OD₆₅₀>0.1). To confirm the MBEC, 5 μL of the recovery media was spotted on LB agar plates in incubated at 37° C. without shaking for 48 h.

### Cell Culture and In Vitro Cytotoxicity Assay

Human lung adenocarcinoma cell line (A549) was maintained in DMEM (Dulbecco's modified Eagle's medium) supplemented with 10% fetal bovine serum (FBS), 1% penicillin. The cells were incubated in 5% CO₂ humidified at 37° C. for growth. The cytotoxicity induced by AuNC@CPP was investigated by 3-(4,5-dimethylthiazol-2-yl)-2,5-diphenyltetrazolium bromide (MTT) assay. A549 cells (2×10⁴/ml, 100 μl/well) were seeded in 96 well plates. After 24 h, cells culture were exposed to concentrations of AuNC@CPP ranging from 0 μg/mL to 3200 μg/mL (AuNC@CPP were dispersed in DMEM). After 24 h incubation, medium containing AuNC@CPP was removed, cells were washed with PBS and incubated with fresh cell culture medium to another 24 h. Then, 20 μl of the MTT (5 mg/mL) was added to each well and incubated for 4 h in 5% CO₂ humidified at 37° C. The medium was removed carefully and 200 μg/L of dimethyl sulfoxide (DMSO) was added to each well to dissolve formazan crystals. Absorbance of formazan was read at 595 nm using the microplate reader. A blank solution (0 μg/mL of AuNC@CPP) was tested and no cytotoxicity could be observed. Three independent experiments and 4 replicates were performed. Results were analyzed as the average of viability (% of the untreated control)±Standard deviation (SD).

### In Vivo Toxicity Evaluation

All animal work was approved by Stanford University's Administrative Panel on Laboratory Animal Care. The 10-12-week-old C57BL/6J mice were purchased from Jackson Laboratories (Sacramento, Calif.) and housed in Stanford University's animal resources facility according to standard guidelines in which food and water were provided ad libitum with the room maintained in 12-hour dark/light cycles. C57BL/6J mice female (n=4 each group) and male (n=3 each group) were divided into 2 groups, control (phosphate-buffered saline, PBS) and AuNC@CPP. The treatments were administrated by oral gavage at a dose of 10 mg/kg (i.e., 1000 mg/mL) daily for 14 days. During the 35-day study, the body weights of animals were measured every two days. At 35 days of post-treatment, mice were sacrificed. Blood and organs were collected. All organs were preserved, fixed in 10% neutral formalin buffer, processed into paraffin embedding, and stained with hematoxylin and eosin for pathology analysis using a light microscope. The microscopic analysis of tissue (histopathology), including esophagus, stomach, heart, kidney, spleen, pancreas, thymus, small intestine, colon, bladder, testis and ovary were carried out for potential histological alteration. Results of the organ-to-body weight ratio (relative organ weight, ROW) were analyzed as the ratio of the organ weight (mg) to the body weight of the animal at necropsy day (g). The following organs were examined for ROW: thymus, spleen, heart, kidneys, liver and testis. Blood samples were subjected to toxicity analysis. An inferior vena cava blood collection was performed at sacrifice. 150 μl of blood was placed in a K₂EDTA tube for hematology analysis and the left blood sample was placed in a 1.5 ml Eppendorf tube for serum extraction. Serum was separated by centrifuging the blood to remove the cellular fraction for liver and renal function testing.

### In Vivo Efficacy Testing

A validate mouse model of P. aeruginosa CSOM to test potential therapeutics was used. Briefly, PA01 was incubated at 37° C. with shaking. After 30 h of incubation, the stationary phase culture was treated with FLOXIN®Otic (final concentration of ofloxacin=5 μg/mL) for 4 h to select persister cells. After creating a subtotal tympanic membrane perforation, we inoculate persister cells (5 μL, 1.6×10⁸ CFU/mL) into middle ear cavity and the mice are allowed to rest with the ipsilateral ear up until recovery. Infection was allowed to develop for 14 days before commencement of antibiotic therapy. All therapy were delivered by placing 8 μL drop in the ear canal twice a day for 14 consecutive days. Therapy outcome was measurement of the bacterial colonies (CFU/mL) from effusion of the middle ear 14 days after stopping treatment.

### Production of Hydroxyl Radical Via the Decomposition of Hydrogen Peroxide by AuNC@CPP

The oxidation of peroxidase substrate 3,3′,5,5′-tetramethylbenzidine (TMB) by hydroxyl radical from the decomposition of hydrogen peroxide (H₂O₂) catalyzed by AuNC@CPP was carried out. The hydroxyl radical is able to oxidize TMB into oxidized TMB (Ox-TMB) with an absorption peak at 652 nm. Thus, the level of hydroxyl radical can be indirectly determined by measuring the absorbance at 652 nm. 50 μL of AuNC@CPP with different concentrations were added into 450 μL 10 mM phosphate buffer (pH 4 and 7.0) containing 0.2 mM TMB and 100 mM of H₂O₂ (30% solution). The mixed solutions were incubated for 30 min at room temperature and absorbance of blue colored product was read at 652 nm using a microplate reader (spectramMax M2, Molecular Devices, Downington, Pa.).

### RecA ATP Hydrolysis (ATPase) Assays

RecA ATPase activity was measured using coupled spectrophotometric enzyme assay. The wild-type RecA, (3 μM) was pre-incubated with ATP and 5 μm poly(dT) for 5 min before APTase measurements. Reactions contained 25 mM Tris-OAc (pH 7.5, 80% cation), 10 mM MgOAc, 2 mM ATP, 3 mM potassium glutamate, 5% w/v glycerol, 1 mM dithiothreitol (DTT), 3 mM PEP, 30 U/ml pyruvate kinase, 30 U/ml lactate dehydrogenase, 4.5 mM NADH and 5 μM poly(dT). Conversion of NADH to NAD⁺ was monitored at 380 nm using a microplate reader (spectramMax M2, Molecular Devices, Downington, Pa.). NADH extinction coefficient of 1.21 mM⁻¹ cm⁻¹ was used to calculate rates of ATP hydrolysis.

### Statistical Analysis

Data are presented as mean±standard deviation. In case of significance the data were tested using unpaired (independent) T-test in GraphPad Prism to show which groups are significantly different from each other.

TABLE 1
Comparison of the antimicrobial efficacy of the various treatments
measured in number of bacterial colonies from an effusion
of the middle ear two weeks after the treatment.

| | Effusion | |
|---|---|---|
| Treatments | (Log₁₀ CFU) | Middle ear mucosa sample |
| PBS | 7.69 ± 0.26 | Culture positive bacteria |
| | (n = 4) | |
| FLOXIN ®Otic | 6.98 ± 1.45 | Culture positive bacteria |
| | (n = 6) | |
| Combination | 2.54 ± 0.51 | Culture positive bacteria |
| | (n = 4) | |

TABLE 2
Erythrocytes and related parameters

| | Mean Value ± SD | | | | | |
|---|---|---|---|---|---|---|
| | Male | | | Female | | |
| Index | PBS | AuNC@CPP | P value | PBS | AuNC@CPP | P value |
| RBC (M/uLI) | 8.50 ± 0.79 | 8.23 ± 1.05 | P > 0.05 | 8.12 ± 1.64 | 7.10 ± 0.73 | P > 0.05 |
| HGB (gm/dL) | 12.40 ± 0.80 | 12.47 ± 1.05 | P > 0.05 | 11.65 ± 2.26 | 10.83 ± 0.68 | P > 0.05 |
| MCV (fL) | 48.75 ± 2.27 | 48.60 ± 1.65 | P > 0.05 | 46.65 ± 1.56 | 48.35 ± 1.70 | P > 0.05 |
| MCH (pg) | 14.60 ± 0.51 | 15.20 ± 0.99 | P > 0.05 | 14.38 ± 0.10 | 15.28 ± 0.83 | P > 0.05 |
| MCHC (g/dL) | 30.00 ± 0.95 | 31.27 ± 1.36 | P > 0.05 | 30.83 ± 0.80 | 31.68 ± 0.84 | P > 0.05 |

RBC: Red blood cell;
HGB: Hemoglobin;
MCV: Mean corpuscular volume;
MCH: Mean cell haemoglobin;
MCHC: Mean corpuscular hemoglobin concentration
P values versus PBS less than 0.05 were considered to be statistically significant.
Data are presented as mean ± s.d.

TABLE 3
Leucocytes and related parameters

| | Mean Value ± SD | | | | | |
|---|---|---|---|---|---|---|
| | Male | | | Female | | |
| Index | PBS | AuNC@CPP | P value | PBS | AuNC@CPP | P value |
| WBC (K/uL) | 3.82 ± 1.13 | 5.24 ± 1.98 | P > 0.05 | 2.70 ± 2.04 | 2.70 ± 1.51 | P > 0.05 |
| NEU (%) | 9.50 ± 0.58 | 6.00 ± 1.00 | P > 0.05 | 10.00 ± 2.58 | 9.25 ± 4.19 | P > 0.05 |
| MON (%) | 3.50 ± 1.00 | 5.00 ± 0.00 | P > 0.05 | 3.75 ± 1.71 | 3.00 ± 1.15 | P > 0.05 |
| LYM (%) | 85.00 ± 2.00 | 87.33 ± 0.58 | P > 0.05 | 85.5 ± 1.00 | 87.00 ± 3.74 | P > 0.05 |
| EOS (%) | 2.00 ± 1.16 | 1.67 ± 0.58 | P > 0.05 | 0.75 ± 0.50 | 0.75 ± 0.96 | P > 0.05 |
| Basophils | 0 | 0 | P > 0.05 | 0 | 0 | P > 0.05 |
| Platelet | 1050 ± 377 | 934 ± 518.16 | P > 0.05 | 649.5 ± 678.17 | 336.75 ± 174.88 | P > 0.05 |
| Count (K/uL) | | | | | | |
| MPV (%) | 6.20 ± 0.06 | 6.30 ± 0.30 | P > 0.05 | 5.9 ± 0.14 | 6.35 ± 0.26 | P > 0.05 |

WBC: White blood cells;
NEU: Neutrophils;
MON: Monocytes;
LYM: Lymphocytes;
EOS: Eosinophils;
MPV: Mean platelet volume
P values versus PBS less than 0.05 were considered to be statistically significant.
Data are presented as mean ± s.d

TABLE 4
Liver function related parameters

| | Mean Value ± SD | | | | | |
|---|---|---|---|---|---|---|
| | Male | | | Female | | |
| Index | PBS | AuNC@CPP | P value | PBS | AuNC@CPP | P value |
| AST (U/L) | 40.33 ± 2.89 | 37.33 ± 2.89 | P > 0.05 | 49.75 ± 4.03 | 61.25 ± 9.64 | P > 0.05 |
| ALT (U/L) | 23.33 ± 5.77 | 26.00 ± 2.65 | P > 0.05 | 23 ± 3.92 | 30.00 ± 4.69 | P > 0.05 |
| ALP (IU/L) | 67 ± 10.4 | 68.33 ± 8.08 | P > 0.05 | 125.5 ± 4.79 | 118.75 ± 10.78 | P > 0.05 |
| TBil (mg/dL) | 0.2 ± 0 | 0.2 ± 0 | P > 0.05 | 0.15 ± 0.06 | 0.23 ± 0.05 | P > 0.05 |

ALT: Alanine transaminase;
AST: Aspartate transaminase;
ALP: Alkaline phosphatase;
Tbil: Toatal Bilirubin
P values versus PBS less than 0.05 were considered to be statistically significant.
Data are presented as mean ± s.d

TABLE 5
Kidney function related parameters

| | Mean Value ± SD | | | | | |
|---|---|---|---|---|---|---|
| | Male | | | Female | | |
| Index | PBS | AuNC@CPP | P value | PBS | AuNC@CPP | P value |
| CR (mg/dL) | 0.197 ± 0.03 | 0.22 ± 0.01 | P > 0.05 | 0.20 ± 0.06 | 0.21 ± 0.03 | P > 0.05 |
| BUN (mg/dL) (mg/dL) (mg/dL) | 39.67 ± 6.51 | 88.5 ± 5.5 | P > 0.05 | 41.5 ± 7.05 | 34.25 ± 5.32 | P > 0.05 |

BUN: Blood urea nitrogen;
CR: Creatinine;
P values versus PBS less than 0.05 were considered to be statistically significant.
Data are presented as mean ± s.d

### REFERENCES

1. Davies, D. Understanding biofilm resistance to antibacterial agents. Nature reviews Drug discovery 2, 114-122 (2003).
2. Koo, H., Allan, R. N., Howlin, R. P., Stoodley, P. & Hall-Stoodley, L. Targeting microbial biofilms: current and prospective therapeutic strategies. Nature Reviews Microbiology 15, 740 (2017).
3. Wolcott, R. et al. Chronic wounds and the medical biofilm paradigm. Journal of wound care 19, 45-53 (2010).
4. Lewis, K. Persister cells, dormancy and infectious disease. Nature Reviews Microbiology 5, 48-56 (2007).
5. Lewis, K. Riddle of biofilm resistance. Antimicrobial agents and chemotherapy 45, 999-1007 (2001).
6. Fisher, R. A., Gollan, B. & Helaine, S. Persistent bacterial infections and persister cells. Nature Reviews Microbiology 15, 453 (2017).
7. Levin-Reisman, I. et al. Antibiotic tolerance facilitates the evolution of resistance. Science 355, 826-830 (2017).
8. Barrett, T. C., Mok, W. W., Murawski, A. M. & Brynildsen, M. P. Enhanced antibiotic resistance development from fluoroquinolone persisters after a single exposure to antibiotic. Nature communications 10, 1-11 (2019).
9. Tolker-Nielsen, T. Pseudomonas aeruginosa biofilm infections: from molecular biofilm biology to new treatment possibilities. Apmis 122, 1-51 (2014).
10. Mulcahy, L. R., Isabella, V. M. & Lewis, K. Pseudomonas aeruginosa biofilms in disease. Microbial ecology 68, 1-12 (2014).
11. Song, J.-J. et al. Changes in antibiotic resistance in recurrent Pseudomonas aeruginosa infections of chronic suppurative otitis media. ENT: Ear, Nose & Throat Journal 95 (2016).
12. Van Acker, H. & Coenye, T. The role of reactive oxygen species in antibiotic-mediated killing of bacteria. Trends in microbiology 25, 456-466 (2017).
13. Kohanski, M. A., Dwyer, D. J. & Collins, J. J. How antibiotics kill bacteria: from targets to networks. Nature Reviews Microbiology 8, 423-435 (2010).
14. Kim, J.-S. et al. Bacterial persisters tolerate antibiotics by not producing hydroxyl radicals. Biochemical and biophysical research communications 413, 105-110
15. Mok, W. W. & Brynildsen, M. P. Timing of DNA damage responses impacts persistence to fluoroquinolones. Proceedings of the National Academy of Sciences 115, E6301-E6309 (2018).
16. Ciofu, O., Rojo-Molinero, E., Macia, M. D. & Oliver, A. Antibiotic treatment of biofilm infections. Apmis 125, 304-319 (2017).
17. Carson, C. & Naber, K. G. Role of fluoroquinolones in the treatment of serious bacterial urinary tract infections. Drugs 64, 1359-1373 (2004).
18. Bassetti, M., Vena, A., Croxatto, A., Righi, E. & Guery, B. How to manage Pseudomonas aeruginosa infections. Drugs in context 7 (2018).
19. Owens Jr, R. C. & Ambrose, P. G. Clinical use of the fluoroquinolones. Medical Clinics of North America 84, 1447-1469 (2000).
20. Zhao, X. et al. Lethal action of quinolones against a temperature-sensitive dnaB replication mutant of Escherichia coli. Antimicrobial agents and chemotherapy 50, 362-364 (2006).
21. Vrany, J. D., Stewart, P. S. & Suci, P. A. Comparison of recalcitrance to ciprofloxacin and levofloxacin exhibited by Pseudomonas aeruginosa bofilms displaying rapid-transport characteristics. Antimicrobial Agents and Chemotherapy 41, 1352-1358 (1997).
22. Olson, M. E., Ceri, H., Morck, D. W., Buret, A. G. & Read, R. R. Biofilm bacteria: formation and comparative susceptibility to antibiotics. Canadian journal of veterinary research 66, 86 (2002).
23. Ceri, H. et al. in Methods in enzymology, Vol. 337 377-385 (Elsevier, 2001).
24. Masadeh, M. M., Alzoubi, K. H., Ahmed, W. S. & Magaji, A. S. In vitro comparison of antibacterial and antibiofilm activities of selected fluoroquinolones against Pseudomonas aeruginosa and methicillin-resistant Staphylococcus aureus. Pathogens 8, 12 (2019).
25. Ohyama, M. et al. Ofloxacin otic solution in patients with otitis media: an analysis of drug concentrations. Archives of Otolaryngology—Head & Neck Surgery 125, 337-340 (1999).
26. Brooun, A., Liu, S. & Lewis, K. A dose-response study of antibiotic resistance in Pseudomonas aeruginosa biofilms. Antimicrobial agents and chemotherapy 44, 640-646 (2000).
27. Moker, N., Dean, C. R. & Tao, J. Pseudomonas aeruginosa increases formation of multidrug-tolerant persister cells in response to quorum-sensing signaling molecules. Journal of bacteriology 192, 1946-1955 (2010).
28. Mulcahy, L. R., Burns, J. L., Lory, S. & Lewis, K. Emergence of Pseudomonas aeruginosa strains producing high levels of persister cells in patients with cystic fibrosis. Journal of bacteriology 192, 6191-6199 (2010).
29. Hall-Stoodley, L. et al. Direct detection of bacterial biofilms on the middle-ear mucosa of children with chronic otitis media. Jama 296, 202-211 (2006).
30. Yao, Q. et al. Understanding seed-mediated growth of gold nanoclusters at molecular level. Nature communications 8, 1-11 (2017).
31. Michael, B. et al. Evaluation of organ weights for rodent and non-rodent toxicity studies: a review of regulatory guidelines and a survey of current practices. Toxicologic pathology 35, 742-750 (2007).
32. Monasta, L. et al. Burden of disease caused by otitis media: systematic review and global estimates. PloS one 7 (2012).
33. Li, M. G., Hotez, P. J., Vrabec, J. T. & Donovan, D. T. Is chronic suppurative otitis media a neglected tropical disease? PLoS neglected tropical diseases 9 (2015).
34. Schilder, A. G. et al. Otitis media. Nature Reviews Disease Primers 2, 1-18 (2016).
35. Ta, N. ENT in the context of global health. The Annals of The Royal College of Surgeons of England 101, 93-96 (2019).
36. del Val, E., Nasser, W., Abaibou, H. & Reverchon, S. RecA and DNA recombination: a review of molecular mechanisms. Biochemical Society Transactions 47, 1511-1531 (2019).
37. Yakimov, A. et al. Blocking the RecA activity and SOS-response in bacteria with a short α-helical peptide. Nucleic acids research 45, 9788-9796 (2017).
38. Drees, J. C., Chitteni-Pattu, S., McCaslin, D. R., Inman, R. B. & Cox, M. M. Inhibition of RecA protein function by the RdgC protein from Escherichia coli. Journal of Biological Chemistry 281, 4708-4717 (2006).
39. Sadekuzzaman, M., Yang, S., Mizan, M. & Ha, S. Current and recent advanced strategies for combating biofilms. Comprehensive Reviews in Food Science and Food Safety 14, 491-509 (2015).
40. Liu, Y. et al. Nanotechnology-based antimicrobials and delivery systems for biofilm-infection control. Chemical Society Reviews 48, 428-446 (2019).
41. Teirlinck, E. et al. Laser-induced vapour nanobubbles improve drug diffusion and efficiency in bacterial biofilms. Nature communications 9, 1-12 (2018).
42. Chua, S. L. et al. Dispersed cells represent a distinct stage in the transition from bacterial biofilm to planktonic lifestyles. Nature communications 5, 1-12 (2014).
43. Fleming, D. & Rumbaugh, K. The consequences of biofilm dispersal on the host. Scientific reports 8, 1-7 (2018).
44. Miyaue, S. et al. Bacterial memory of persisters: bacterial persister cells can retain their phenotype for days or weeks after withdrawal from colony—biofilm culture. Frontiers in microbiology 9, 1396 (2018).
45. Soares, A. et al. Understanding Ciprofloxacin Failure in Pseudomonas aeruginosa Biofilm: Persister Cells Survive Matrix Disruption. Frontiers in Microbiology 10, 2603 (2019).
46. Mo, C. Y. et al. Systematically altering bacterial SOS activity under stress reveals therapeutic strategies for potentiating antibiotics. MSphere 1, e00163-00116 (2016).

### Example 2

AuNC@CPP Restores Antibiotic Susceptibility of Persister Cells of Pseudomonas Aeruginosa from a Cystic Fibrosis Patient

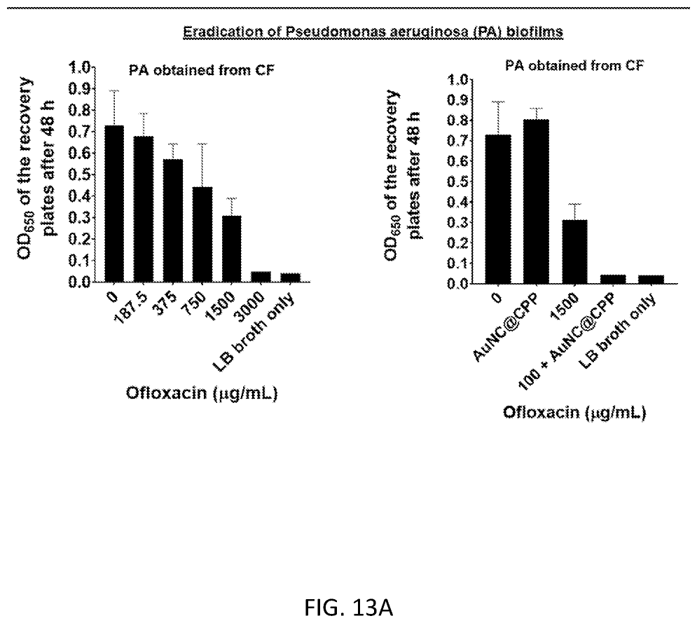

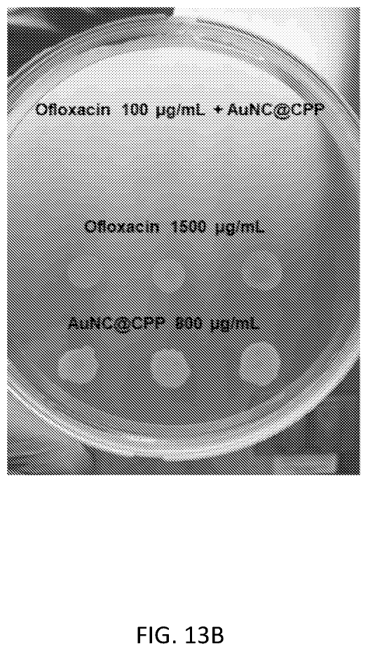

The biofilm eradication capacities of ofloxacin, AuNC@CPP (800 μg/mL), or a combination of ofloxacin and AuNC@CPP were tested on Pseudomonas Aeruginosa from a cystic fibrosis (CF) patient (FIGS. 13A-13B). Following treatment, optical density (OD) was measured from recovery plates after 48h incubation (650 nm). Nanoparticle treatment worked in treating pseudomonas associated with cystic fibrosis, highlighting its utility against any chronic biofilm infection.

### Example 3

Demonstrating AuNC@CPP does not Act Through Efflux Pump Inhibition

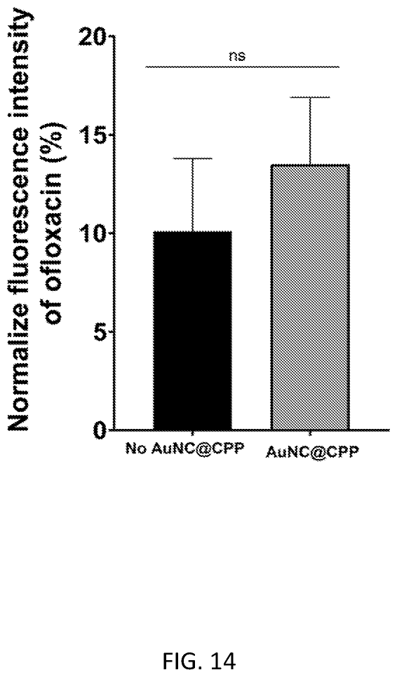

Current nano-adjuvants for fluoroquinolone based on cationic hydrophobically functionalized gold nanoparticles act as efflux pump inhibitors. Our AuNC@CPP are anionic hydrophilically functionalized gold nanoparticles covalently conjugated with CPP to enhance intracellular delivery into persister cells. The mechanism of action of an efflux pump inhibitor is through competitive inhibition resulting in an increase in intracellular antibiotic concentration. We evaluated the intracellular OFL concentration in stationary phase planktonic PA populations after 30 min incubation with OFL (10 μg/mL) in the absence and presence of AuNC@CPP using a previously described spectrofluorometric method (limit of quantitation 24 ng/mL) (45). Co-administration of AuNC@CPP plus OFL did not significantly increase the intracellular concentration of OFL, suggesting that the synergy of our combination therapy cannot be attributed to the ability of AuNC@CPP to act as an efflux pump inhibitor (FIG. 14). Although not wishing to be bound by theory, a plausible mechanism is that AuNC@CPP acts both as an enhancer of ROS-mediated OFL lethality and inhibits the repair of bacterial DNA.

### Example 4

### Demonstrating In Vitro Efficacy

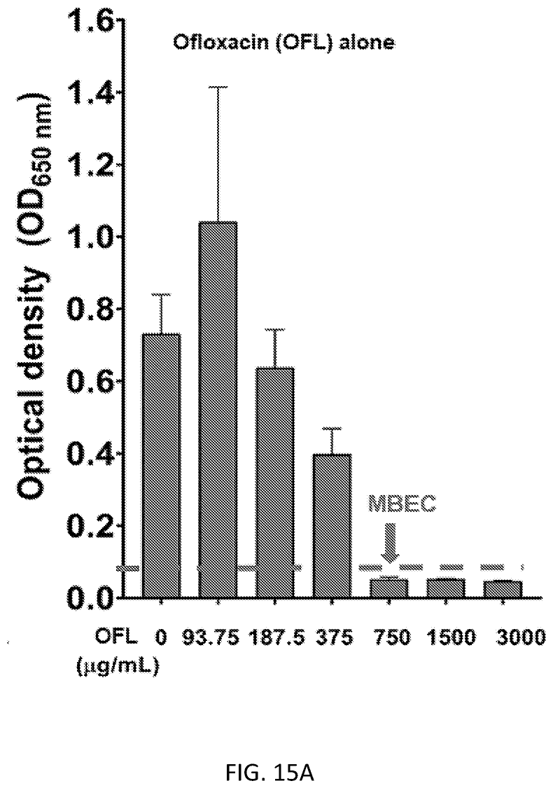

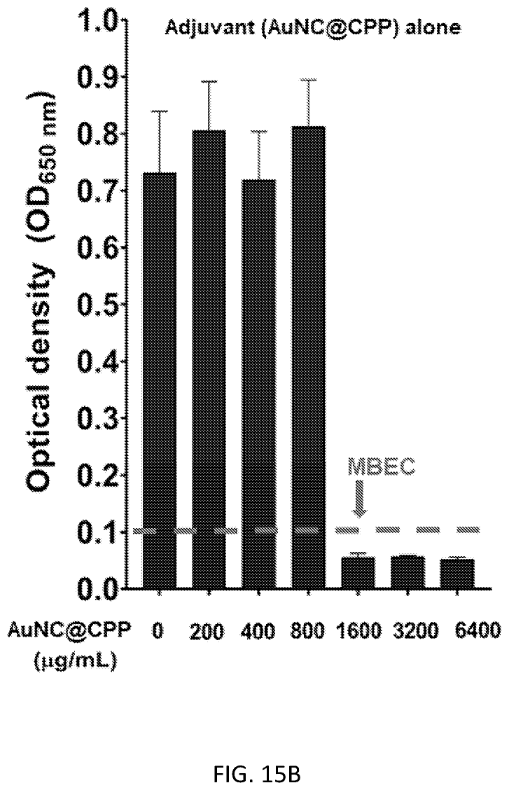

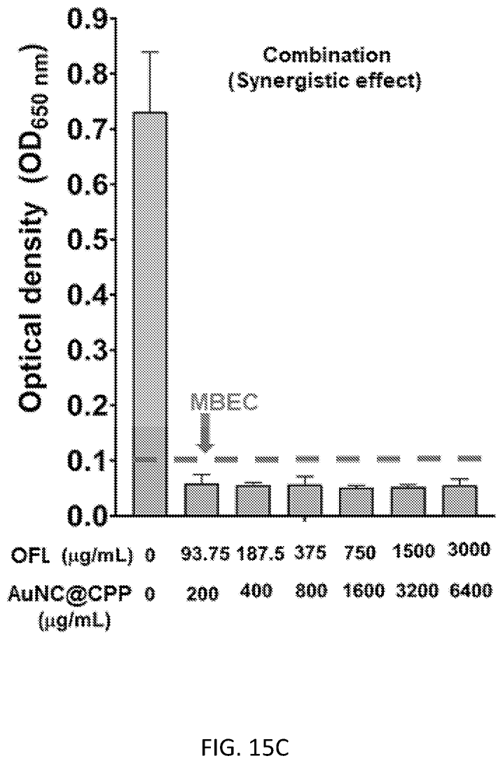

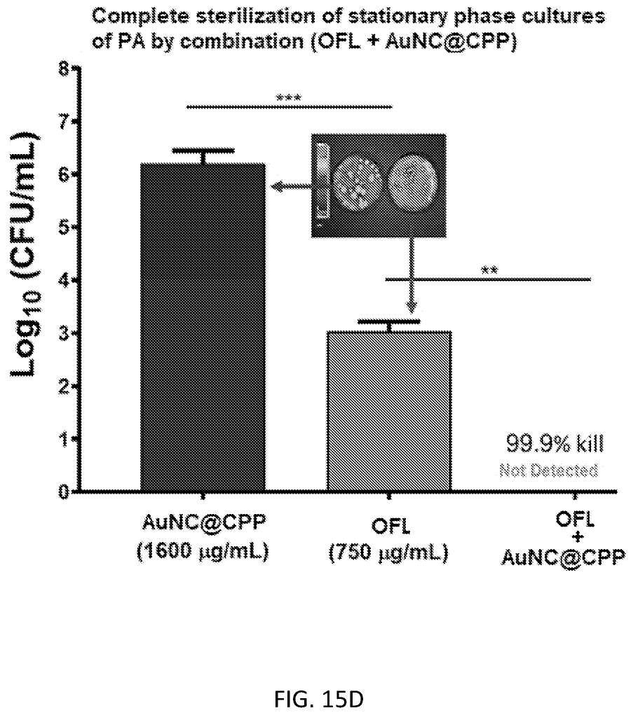

We tested the eradication potential of OFL, AuNC@CPP, and their combination against both biofilms and stationary phase planktonic PA (chromosomally encoded luminescent PA01) in vitro. Biofilms of PA01 (10-day-old) were grown in the commercially available Calgary Biofilm Device (a 96-well plate)/minimum biofilm eradication concentration (MBEC) assay plate (46). Spent media was removed and biofilms were treated with serial dilutions of OFL, AuNC@CPP, and their combination and incubated for 24 hr. Wells were washed with PBS and biofilms were disrupted with sonication and incubated in recovery media for turbidity measurements. A well was considered to have biofilm eradicated if the optical density at 650 nm (OD₆₅₀) was less than 0.1 after 24 hours. As expected, OFL and AuNC@CPP had little effect on the PA biofilms with a MBEC of 750 and 1600 μg/mL, respectively (FIGS. 15A-15D). The combination of AuNC@CPP plus OFL resulted in eradication of persister cells in the biofilm (OD650<0.1). Elimination of the PA biofilm is unprecedented for such a low MBEC (93.75 μg/mL OFL), a clinically achievable concentration of OFL in middle ear mucosa (47). Importantly, the combination of AuNC@CPP (16000 μg/mL) plus OFL (750 μg/mL) was able to effectively sterilize a culture of stationary phase planktonic PA. There were no remaining CFU (99.99% killing) compared to OFL (CFU=10³) and AuNC@CPP (CFU=10⁶) after 24 hr treatment. Eradication of biofilms and stationary phase planktonic PA indicates that our AuNC@CPP combined with OFL therapy could eradicate PA CSOM in vivo.

### Example 5

### AuNC as a Stand-Alone Therapy

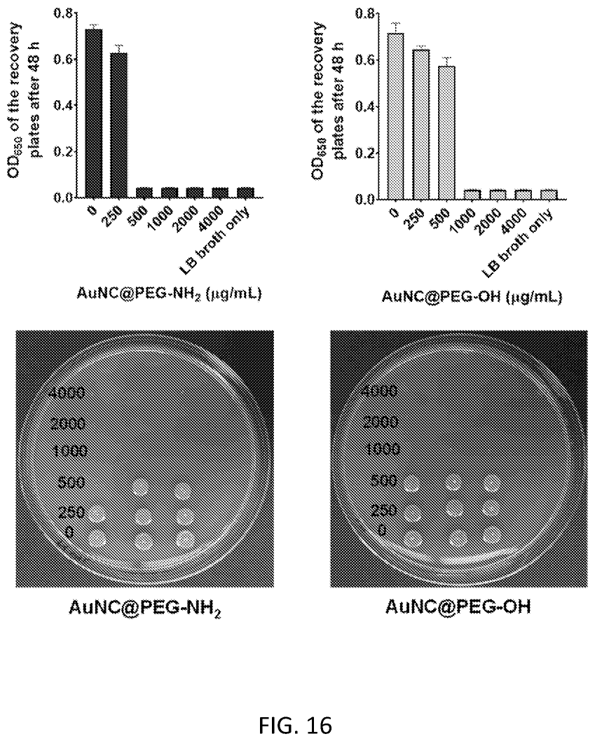

Although the OFL and AuNC@CPP worked better in synergy, AuNC@CPP also was effective alone if administered at high enough concentrations. Viability of an E. coli biofilm was determined after treatment with AuNC@PEG-NH₂ or AuNC@PEG-OH for 24 h (FIG. 16). Following treatment, optical density (OD) from recovery plates after 48 h incubation was measured at 650 nm. Wells with an OD650 of ≤0.1 provided evidence of biofilm eradication. The minimum E. coli biofilm eradication concentration (MBEC) value of both AuNCs is 1 mg/mL.

### Example 6

### AuNC@CPP Effectiveness in Biofilm Eradication of Gram-Positive Bacteria.

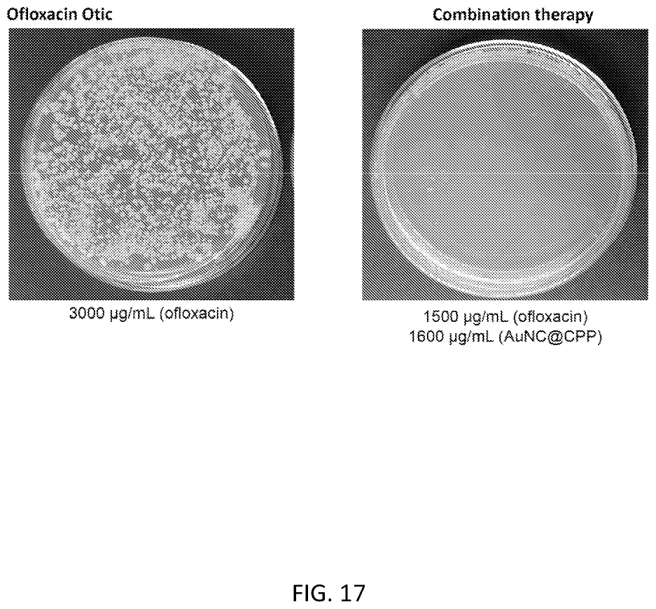

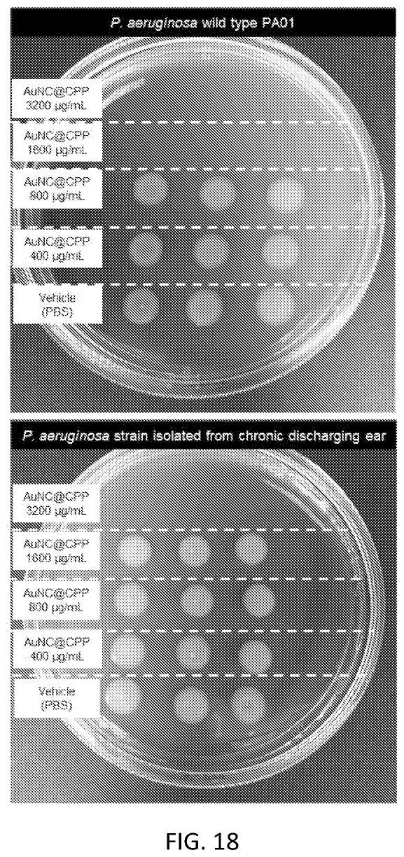

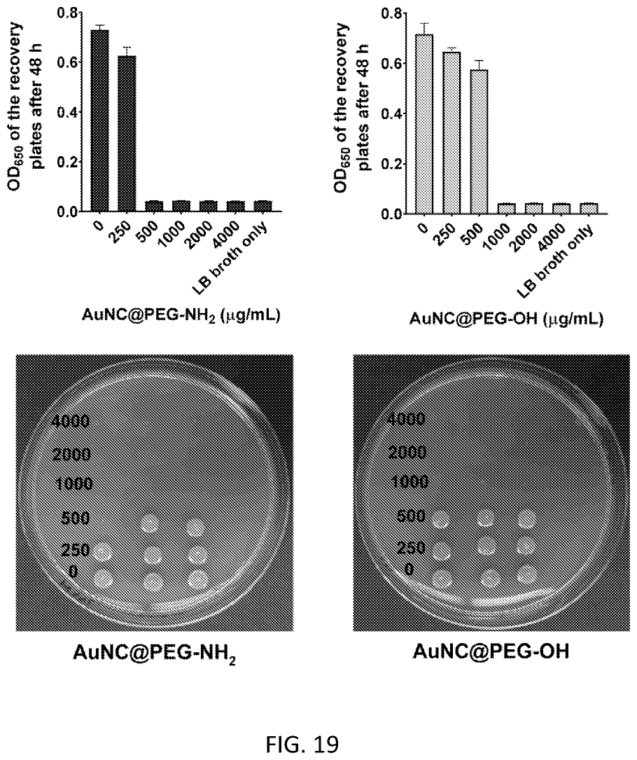

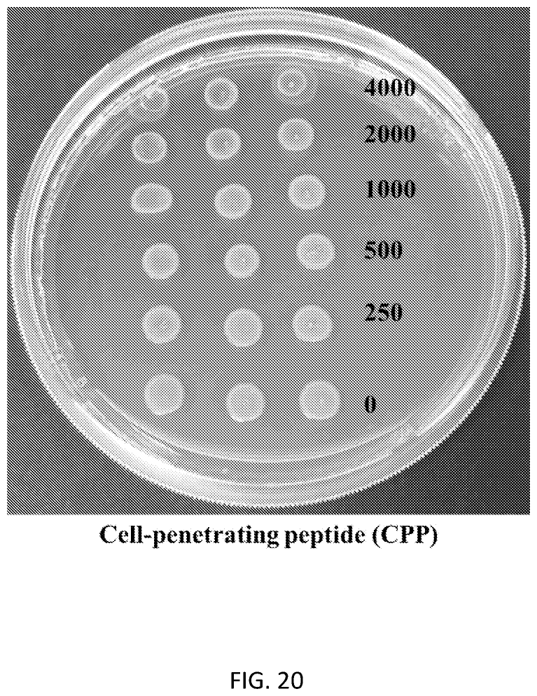

Ofloxacin (OFL) by itself at concentrations of 3000 μg/ml was unable to eradicate biofilms of a Staphylococcus aureus (SA) clinical isolate from CSOM SA. However, OFL combined with AUNC@CPP was effective at eradicating SA biofilms (FIG. 17). These results show that the AuNC@CPP also works on biofilms of gram-positive bacteria (SA), not just gram-negative (PA) bacteria.

### Example 7

### AuNC@CPP Effectiveness in Eradication of a Meropenem Resistant Strain of Klebsiella

We took a Meropenem-resistant strain of Klebsiella with MIC>200 and MBC>200 and overcame its resistance by bringing down the MIC to 1 μg/ml and MBC to 2 μg/ml when treated with Meropenem in combination with AuNC@CPP (see Table 6). This highlights the ability of the AuNC@CPP to not only treat antimicrobial resistance in biofilms, but also antimicrobial resistance generally, including sepsis and other acute infections.

TABLE 6
Minimum Inhibitory Concentration (MIC) and Minimum Bactericidal Concentration
(MBC) for AuNC@CPP alone or in Combination with Meropenem

| | | | | | AuNC@CPP + | |
|---|---|---|---|---|---|---|
| | | | AuNC@CPP | | Meropenem | |
| | | | MIC | MBC | MIC | MBC |
| Species | Strain ID | Characteristics | (μg/ml) | (μg/ml) | (μg/ml) | (μg/ml) |
| Klebsiella | CDC | Carbapenem | >200 | >200 | 50 + 1 | 100 + 2 |
| pneumoniae | 0044 | resistant, | >200 | >200 | 50 + 1 | 100 + 2 |
| | | ESBL | | | | |
| | | producing | | | | |

AuNC@CPP is coated with carboxylic acid-functionalized polyethylene glycol (PEG-COOH).

1. A nanoparticle having a size of less than 10 nm in length that is functionalized with an anionic moiety and a cell penetrating peptide, wherein the anionic moiety and the cell penetrating peptide are attached to the outer surface of the nanoparticle.

2. The nanoparticle of claim 1, wherein the cell-penetrating peptide is a human immunodeficiency virus (HIV) trans-activator of transcription (TAT) cell-penetrating peptide

3. The nanoparticle of claim 2, wherein the TAT cell-penetrating peptide comprises the amino acid sequence of SEQ ID NO:7.

4. The nanoparticle of claim 1, wherein the anionic moiety comprises a carboxylate functional group, a phosphate functional group, or a sulfate functional group.

5. The nanoparticle of claim 1, further comprising a polyethylene glycol (PEG) polymer, wherein the PEG polymer is attached to the outer surface of the nanoparticle.

6. The nanoparticle of claim 5, wherein the PEG polymer is functionalized with the anionic moiety.

7. The nanoparticle of claim 6, wherein the PEG polymer is polyethylene glycol carboxylic acid (PEG-COOH) or thiol-carboxyl polyethylene glycol (COOH-PEG-SH).

8. The nanoparticle of claim 1, further comprising an antimicrobial agent selected from the group consisting of a D-carbohydrate, a D-amino acid, a nucleic acid comprising a CrcZ RNA sequence, and a nucleic acid comprising a CrcZ A-rich motif sequence, wherein the antimicrobial agent is attached to the outer surface of the nanoparticle.

9. (canceled)

10. The nanoparticle of claim 8, wherein the CrcZ RNA sequence comprises:
a) a nucleotide sequence of SEQ ID NO:1;
b) a nucleotide sequence having at least 90% identity to the sequence of SEQ ID NO:1, wherein the nanoparticle is capable of rendering a persister cell susceptible to an antibiotic; or
c) an RNA equivalent of a) or b).

11. The nanoparticle of claim 8, wherein the CrcZ A-rich motif sequence comprises:
a) ACAACAACAATAACAA (SEQ ID NO:2);
b) CAATAAGAA;
c) AACAAGAACAA (SEQ ID NO:3);
d) AGAACAACAAAA (SEQ ID NO:4);
e) ACAACAAGAACAA (SEQ ID NO:5);
f) AGAACAAGAACAA (SEQ ID NO:6);
g) AACAACAA;
h) AAAAACAA; or
i) an RNA equivalent of a)-i).

12. The nanoparticle of claim 1, wherein the nanoparticle further comprises a linker connecting the antimicrobial agent or the cell penetrating peptide to the outer surface of the nanoparticle.

13-15. (canceled)

16. The nanoparticle of claim 1, wherein the nanoparticle comprises a metal, a ceramic, graphite or other carbon-based material, or silica.

17. (canceled)

18. A composition for treating an infection comprising the nanoparticle of claim 1 and a pharmaceutically acceptable excipient or carrier.

19. (canceled)

20. The composition of claim 18, further comprising an antibiotic.

21-22. (canceled)

23. A method of treating an infection in a subject, the method comprising administering a therapeutically effective amount of the composition of claim 18 to the subject.

24. The method of claim 23, further comprising administering a therapeutically effective amount of at least one antibiotic in combination with the composition.

25. The method of claim 23, wherein the subject has a chronic infection.

26. The method of claim 25, wherein the infection is an ear infection, a cutaneous infection, or a lung infection.

27-38. (canceled)

39. The method of claim 23, wherein the composition is administered locally at the site of infected tissue.

40-45. (canceled)

46. A method of eradicating bacteria in a biofilm, the method comprising contacting the biofilm with an effective amount of the nanoparticle of claim 1.

47. (canceled)

48. The method of claim 46, wherein the biofilm is on a medical device, a personal hygiene article, toiletry, cosmetic, disinfectant, cleaning solution, or in a water treatment or distribution system.

49-52. (canceled)
